SEQUENCE LISTING (87 sequences)

SEQ ID NO: 1 - protein, 529 aa, Eleusine indica
MVATPAMAAAAPPLRAPRFHARHRRRSVRCAVASDATEAPAAPGARLSADCVVVGGGISGLCTAQALATKHGIGDVLVTEARARPGGNITTVERPDEGYLWEEGPNSFQPSDPVLTMAVDSGLKEDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEEAGGSIIGGTIKTIQERGKNPKPERDPRLPKPKGQTVASFRKGLAMLPNAITSRLGSKVKLSWKLTSITKSDSKGYVLVYETPEGIVSVQAKSVIMTIPSYVASDILRPLSSDAADALSRFYYPPVAAVTVSYPKEAIRKECLIDGELQGFGQLHPRSQGVETLGTIYSSSLFPNRAPAGRVLLLNYIGGATNTGIVSKSESELVEAVDRDLRKMLINPRAVDPYVLGVRVWPQAIPQFLVGHLDILDAAKSALNRSGYDGLFLGGNYVAGVALGRCVEGAYESASQISDFLTKYAYK

SEQ ID NO: 2 - DNA, 3816 bp, Eleusine indica
  misc_feature (689)..(689): n is a, c, g, or t
atggtcgccacgcccgcaatggccgccgccgcgccgccgctccgagcgccgcgattccatgcgcgtcaccgccgcagaagcgtgcgctgcgcggtggccagcgacgccaccgaggcgccggccgcgcccggcgcgcggctgtccgcggactgcgtcgtggtgggcggcggcatcagcggcctctgcacggcgcaggcgctggccacgaaacacggcatcggagacgtacttgtcacggaggcccgcgcccgccccggcggcaacatcaccaccgtcgagcgccccgacgaggggtacttatgggaggaagggcccaacagcttccagccctccgaccccgtcctcaccatggccgtacgcttctcgctcccttcttctattcctcttacagtacatttctcgtgaacgctgaatgaactgggcgcgcgcgcgcaggtggacagcggactcaaggaggacttggtgtttggagacccgaacgcgccgaggttcgtcctgtgggaagggaagctgaggccggtgccgtccaagcccgccgaccttccgttcttcgatctcatgagcatcccgggcaagctccgggccggccttggcgccctcggcattcgcccgccgcctccagttcgttctctccccaaatgctgcattcgtgattcttctgcattttgattgttcaactgtcattggangttcgttctctccccaaatgctgcattcgtgattcttctgcattttgattgttcaactgtcattggcgctgagcgttactggcaaaggtttcagggccgtgaggagtcggtggaggagtttgtgcgccgcaacctcggtgctgaggtctttgagcgcctcatcgagcctttctgctcaggtgcttattgcattgtacaatggctgttttgttatgatttcctgcttgcagtcattccctataaaagaatgtaacagtttcaatgttaacataaggtgtctatgctggtgatccttcaaaactcagtatgaaggctgcatttgggaaggtatggaggcttgaggaggctggaggtagtattattggtggaacaattaaaacaatccaggagaggggcaagaatcctaagccagagagggatccgtaagagaacaacttacttttcttctgttgcattatctgtctattcttatcattctgatcaattttctccagccgccttccaaagccaaagggacagactgttgcatccttcaggaagggtctcgccatgcttccaaatgccattacatccaggtttattctcatatcatgtatatactatttgtatagcgcatcaactttgcacgagggtgccacagattgtttggatagacgtagtccttcagagacttcgtacatctcgtttgccaaattcattaccatattattgcatcacctcttttcatcctcagtgcatattagttaggttactttcaccctttttcatctcactttatgttgtcatttacaatataggtaaatggtttctagttgtctgtggaaactactaacatatatggcttcagattgtaagggataaatacagattatgcatttattctagtgtgtgtgcttgtgacttcatcaaaagctatgaaaatttgcaattgagatatctgatatagacttatggtactaaccacttaactttgttccattctgatccacctttgtttacaggttgggtagtaaagtcaaactgtcttggaaactcacaagcattacaaaatcagacagcaagggttatgtattggtgtatgaaacaccagaaggaatcgtctcagtccaggctaaaagtgttatcatgaccattccctcctacgttgctagtgacatcttgcgcccactttcagtaagtataaaactaatcaaatttcatgttctgtaaactgaggcactgttagcttcttaattgggaaatgggcacccagcagtagcattcacacttaaggaattcattctttctgccattctgtagtcaacgtctcaatgcagagatgtatagaaacacaaaggaaattgctacaccttgtttgatttgagtaacatctgaatgtttaattttcagaatggatcatctttctgagatatgcataatgtaacttaagcctttcctaattactaaacacaattgaattgctgagattgaagagaagcttgtgcaccatcatttgagatggcttgcacatatccaacagaggcctcaagatgcactggtaatgtgaacaagaggcagaggaagaccaaacttgacattggaggagtctgtgaagagggatatgaaggactggaatatcgttaaggagttagctatagatagagaacgtgtaaaccaataactaacgtgccaaaaccttgattttcggtttcccccatttttacccttttctgtttggtttattattgttcttttcctctccttgtttctttctttttttcctttttgtgtaatctctttgtgtgagtcttgcgggtttcatctctagtctatcccaacttgggactaaaaggttttattgttgttgttgaattcaattgctgagatcagaaggattgaacggaagaaggttatgcgaatggaaatgctttaggtcgagggcaaaagagtcataatattgatgtactagttcggtttggacagcttcttaaccaattaatgttctaggctttcaaataatctgaaacaacaacgttttatgtttctactgttttcctaagtttatatcttgttcgaacaaaaatggacgtagtgatcaattttgataattctatatgttcaggtaactgctatataacttgtcaactacagaattggggttattcaagtatgagagaggagactatgtttagtagcactatacaatttcctagaatttctaaggtaatgtaggcatacttaacttgtttttccataccacagagtgatgctgcagatgctctttcaagattctattatccaccagtcgctgctgtaactgtttcatatccaaaggaagcaattcgaaaagaatgcttaatcgatggggagctccagggtttcggccagttgcatccacgtagtcaaggagttgagacattaggtaaattttcctttttttttacatttgagtcttacgctctgtacagatcattatcattattatgtggcttaatgtttttattgtatttgttaaagtactatttggattgcaaatcgacaatcttattggtgttattcttttcaaaggaacaatatacagctcttcgctctttccgaatcgtgctcctgctggaagggtcttacttcttaactacataggaggtgctacaaatacagggatcgtttccaaggttctattctctgtcaaactgattgatccttatttgttcatgatcttctgatatgtttacaacatcctttactgtgatcttcaaataaaacacatgttactgctgatcacctattcttcattgattgacgtaaatgctgactgcatttttcactactgatctcatcttatttgcagagcgaaagtgagctggtagaagcagttgaccgtgatcttaggaaaatgctcataaatcctagagcagtggatccttatgtccttggtgtgcgggtgtggccacaagctattcctcagttcctggtaggacatcttgatattctcgatgctgcaaaatctgccctcaacagaagtggctacgatgggctattcctgggagggaactatgtagcaggagttgctctggggcgctgcgttgaaggtgcatatgaaagtgcctcacaaatatctgacttcttgaccaagtacgcctacaagtga

SEQ ID NO: 3 - protein, 511 aa, Eleusine indica
MAGSDDTRAAPARSVAVVGAGVSGLAAAYRLRKSGVNVTVFEAGDRAGGKIRSNSEGGFLWDEGANTMTESELEVSRLIDDLGLQDRQQYPNSQHKRYIVKDGAPVLIPSDPISLIKSSVLSTKSKFALFLEPFIYKKTSTRNSGIVSDEHLSESVGSFFERHFGQEVVDYLIDPFVAGTSGGDPESLSIRHAFPALWNLEKKYGSVIAGAILSKLTAKRDPVKKTSDSSGKRRNRRVSFSFLGGMQSLIDALHNEVGDGNVKLSTEVLSLACSVDGVPASGGWSISIDSKNTGSKEFGKKQAFDAVIMTAPLSNVQKMKFMKGGAPFVLDFLPKVDYLPLSLMVTAYKKEDVKRPLEGFGVLIPYKEQQKHGLKTLGTLFSSMMFPDRAPSDQFLYTTFIGGSHNRDLAGAPTTILKQLVTSDLRKLLGVEGQPTFVKHVYWRNAFPLYGRDYNSVLEAIEKMEKNLPGFFYAGNNKDGLAVGSVIASGSKAADLAISYLESRTKHDSSH

SEQ ID NO: 4 - DNA, 1537 bp, Eleusine indica
atggcgggctccgacgacacgcgcgccgctcccgccaggtcggtcgccgtcgtgggcgccggcgtcagtgggctcgcggcggcgtacaggctgcggaagagcggcgtcaacgtgacggtgttcgaggcgggtgacagggccggagggaagatacgaagcaactccgagggtggattcctctgggatgaaggggccaacaccatgacagaaagtgaattggaggtcagcagattaattgatgatcttggtctccaagacagacagcagtatcctaactcccaacacaagcgttacattgtcaaagatggtgcaccagtactgattccttcagatcctatttccttaataaaaagcagtgttctgtctacaaaatcaaagtttgcattatttctagagccatttatttacaagaagactagcacaagaaactctggaatagtgtctgatgagcatttaagtgagagtgttgggagcttctttgaacgccactttggacaagaggtagttgactatcttatcgatccatttgtagctggaacaagtggcggagatcctgagtcattatctattcgacatgcatttccagcactgtggaatttagagaaaaagtatggttctgtcattgctggcgccatcttgtctaaactaactgccaaacgcgatcctgtcaagaaaacaagtgattcatcagggaaaagaaggaataggcgtgtgtcattttcatttcttggaggaatgcagtcactaatagatgcacttcacaatgaagttggagatggtaatgtgaagctcagtacagaagtgttgtccctggcatgtagtgtcgatggtgtgcctgcatccggtgggtggtcaatttctattgattcaaaaaataccggtagcaaggagtttggaaagaaacaagccttcgatgctgtaataatgacagctccattgtctaatgtgcagaagatgaagtttatgaaagggggagctccatttgtgttagactttcttcctaaggtggattatctgccactatccctcatggtgacagcttacaagaaggaagacgtcaagagacctctggaaggatttggggtactaataccctataaggaacagcaaaagcatggcctgaaaactcttggtactctcttctcctcgatgatgttcccagatcgagctcctagtgatcaatttctatatacaactttcattgggggtagccacaatcgagatcttgctggagctccaacgactattctaaagcaacttgtgacatctgaccttagaaagcttttgggtgtggaagggcaaccaacttttgtcaagcatgtatactggagaaatgcttttcctttgtatggccgtgattacaattccgtattggaagctatagagaagatggagaaaaatctaccagggttcttctatgcaggaaataacaaggatgggctggctgttgggagtgttatagcttctggaagcaaggctgctgaccttgcaatttcgtatcttgaatctcgcacaaagcatgacagttcacattgaa

SEQ ID NO: 5 - protein, 1587 aa, Artificial
  Codon-optimized protoporphyrinogen oxidase (PPO1)
ATGGTTGCAACACCTGCTATGGCTGCAGCTGCACCACCTTTGAGGGCACCAAGATTTCATGCTAGGCACAGAAGAAGGTCTGTTAGATGTGCTGTTGCATCAGATGCAACCGAGGCTCCAGCTGCACCTGGTGCAAGGTTGTCTGCTGATTGTGTTGTGGTTGGAGGTGGAATTTCAGGATTGTGCACAGCTCAAGCACTTGCTACCAAGCATGGTATTGGAGATGTGCTTGTTACTGAAGCAAGGGCTAGACCAGGTGGAAACATTACTACAGTGGAGAGACCTGATGAAGGTTATTTGTGGGAGGAAGGACCAAATTCTTTCCAGCCATCAGATCCTGTGCTTACAATGGCTGTTGATTCTGGTTTGAAGGAAGATTTGGTTTTTGGAGATCCAAACGCACCTAGGTTCGTGTTGTGGGAGGGAAAGTTGAGACCAGTTCCTTCTAAGCCAGCTGATTTGCCATTTTTCGATCTTATGTCAATTCCTGGAAAGTTGAGGGCAGGTTTGGGAGCTTTGGGTATTAGGCCACCTCCACCTGGAAGAGAGGAATCTGTGGAGGAATTTGTTAGGAGAAATTTGGGTGCTGAGGTGTTTGAAAGACTTATTGAGCCTTTCTGCTCAGGTGTTTACACCGGAGATCCATCTAAGTTGTCTATGAAGGCTGCATTCGGAAAGGTTTGGAGGCTTGAGGAAGCTGGTGGATCTATTATTGGTGGAACAATTAAGACCATTCAAGAGAGAGGAAAGAACCCAAAGCCTGAAAGAGATCCAAGACTTCCAAAGCCTAAGGGTCAGACCGTGGCTTCTTTTAGAAAGGGATTGGCAATGCTTCCAAATGCTATTACTTCTAGGCTTGGTTCAAAGGTTAAGTTGTCTTGGAAGTTGACTTCAATTACAAAGTCAGATTCTAAGGGTTATGTGCTTGTTTACGAGACTCCAGAAGGAATTGTGTCTGTTCAAGCAAAGTCAGTGATTATGACAATCCCTTCTTATGTTGCTTCAGATATTTTGAGGCCACTTTCTTCAGATGCTGCAGATGCTCTTTCTAGGTTCTATTACCCACCTGTGGCTGCAGTGACTGTTTCATACCCTAAGGAAGCTATTAGGAAGGAATGTTTGATTGATGGAGAGCTTCAAGGTTTTGGACAGCTTCATCCAAGGTCTCAGGGTGTTGAAACCTTGGGAACTATCTATTCTTCTTCTCTTTTCCCAAACAGGGCACCTGCTGGTAGAGTGCTTTTGCTTAACTACATTGGTGGAGCTACCAATACTGGAATTGTTTCAAAGTCTGAGTCAGAATTGGTGGAAGCAGTTGATAGGGATTTGAGAAAGATGCTTATTAATCCAAGGGCTGTTGATCCTTATGTTTTGGGTGTGAGAGTTTGGCCACAAGCAATTCCTCAGTTTCTTGTGGGACACTTGGATATTCTTGATGCTGCAAAGTCTGCTCTTAACAGGTCAGGATACGATGGATTGTTCCTTGGTGGAAATTACGTGGCAGGTGTTGCTTTGGGAAGATGCGTTGAGGGAGCATACGAATCTGCTTCACAAATTTCTGATTTTCTTACAAAGTATGCTTACAAG

SEQ ID NO: 6 - DNA, 20 bp, Artificial
  Synthetically generated oligonucleotide
atggtcgccacgcccgcaat

SEQ ID NO: 7 - DNA, 22 bp, Artificial
  Synthetically generated oligonucleotide
cttgtaggcgtacttggtcaag

SEQ ID NO: 8 - DNA, 20 bp, Artificial
  Synthetically generated oligonucleotide
atggcgggctccgacgacac

SEQ ID NO: 9 - DNA, 23 bp, Artificial
  Synthetically generated oligonucleotide
atgtgaactgtcatgctttgtgc

SEQ ID NO: 10 - protein, 529 aa, Eleusine indica; identical to SEQ ID NO: 1

SEQ ID NO: 11 - protein, 529 aa, Eleusine indica
MVATPAMAAAAPPLRAPRFHARHRRRSVRCAVASDATEAPAAPGARLSADCVVVGGGISGLCTAQALATKHGIGDVLVTEARARPGGNITTVERPDEGYLWEEGPNSFQPSDPVLTMAVDSGLKEDLVFGDPNAPRFVLWEGKLRPVPSKPADLPFFDLMSIPGKLRAGLGALGIRPPPPGREESVEEFVRRNLGAEVFERLIEPFCSGVYTGDPSKLSMKAAFGKVWRLEEAGGSIIGGTIKTIQERGKNPKPERDPRLPKPKGQTVASFRKGLAMLPNAITSRLGSKVKLSWKLTSITKSDSKGYVLVYETPEGIVSVQAKSVIMTIPSYVASDILRPLSSDAADALSRFYYPPVAAVTVSYPKEAIRKECLIDGELQGFGQLHPRSQGVETLGTIYSSSLFPNRAPAGRVLLLNYIGGATNTGIVSKSESELVEAVDRDLRKMLINPRAVDPYVLGVRVWPQAIPQFLVGHLDILDAAKSALNRSGYDGLFLGGNYVAGVALGRCVEGAYESASQISDFLTKYAYK

SEQ ID NO: 12 - protein, 529 aa, Eleusine indica
MVATPAMAAAAPPLRAPRFHARHRRRSVRCAVASDATEAPAAPGARLSADCVVVGGGISGLCTAQALATKHGIGDVLVTEARARPGGNITTVERPDEGYLWEEGPNSFQPSDPVLTMAVDSGLKEDLVFGDPNAPRFVLWEGKLRPVPSKPADLPFFDLMSIPGKLRAGLGALGIRPPPPGREESVEEFVRRNLGAEVFERLIEPFCSGVYTGDPSKLSMKAAFGKVWRLEEAGGSIIGGTIKTIQERGKNPKPERDPRLPKPKGQTVASFRKGLAMLPNAIASRLGSKVKLSWKLTSITKSDSKGYVLVYETPEGIVSVQAKSVIMTIPSYVASDILRPLSSDAADALSRFYYPPVAAVTVSYPMEAIRKECLIDGELQGFGQLHPRSQGVETLGTIYSSSLFPNRAPAGRVLLLNYIGGATNTGIVSKSESELVEAVDRDLRKMLINPRAVDPYVLGVRVWPQAIPQFLVGHLDILDAAKSALNRSGYDGLFLGGNYVAGVALGRCVEGAYESASQISDFLTKYAYK

SEQ ID NO: 13 - protein, 550 aa, Amaranthus tuberculatus
MSAMALSSSILQCPPHSDISFRFFAHTRTQPPIFFGRPRKLSYIHCSTSSSSTANYQNTITSQGEGDKVLDCVIVGAGISGLCIAQALSTKHIQSNLNFIVTEAKHRVGGNITTMESDGYIWEEGPNSFQPSDPVLTMAVDSGLKDDLVLGDPNAPRFVLWNGKLRPVPSKPTDLPFFDLMSFPGKIRAGLGALGLRPPPPSYEESVEEFVRRNLGDEVFERLIEPFCSGVYAGDPAKLSMKAAFGKVWTLEQKGGSIIAGTLKTIQERKNNPPPPRDPRLPKPKGQTVGSFRKGLIMLPTAIAARLGSKVKLSWTLSNIDKSLNGEYNLTYQTPDGPVSVRTKAVVMTVPSYIASSLLRPLSDVAADSLSKFYYPPVAAVSLSYPKEAIRPECLIDGELKGFGQLHPRSQGVETLGTIYSSSLFPGRAPPGRTLILSYIGGATNLGILQKSEDELAETVDKDLRKILINPNAKGSRVLGVRVWPKAIPQFLVGHFDVLDAAKAGLANAGQKGLFLGGNYVSGVALGRCIEGAYDSASEVVDFLSQYKDK

SEQ ID NO: 14 - protein, 535 aa, Setaria italica
MVAAAMATAPSAGVPPLRGTRGPARFRIRGVSVRCAAVAGGAAEAPASAGARVSADCVVVGGGISGLCTAQALATKHGVGDVLVTEARARPGGNITTVERPDEGYLWEEGPNSFQPSDPVLTMAVDSGLKDDLVFGDPNAPRFVLWEGKLRPVPSKPADLPFFDLMSIPGKLRAGFGALGIRPPPPGREESVEEFVRRNLGAEVFERLIEPFCSGVYAGDPSKLSMKAAFGKVWRLEEAGGSIIGGTIKTIQERGKNPKPPRDPRLPTPKGQTVASFRKGLAMLPNAITSSLGSKVKLSWKLTSITKSDGMGYVLVYETPEGVVSVQAKSVIMTIPSYVASDILRPLSSDAADALSRFYYPPVAAVTISYPKEAIRKECLIDGELQGFGQLHPRSQGVETLGTIYSSSLFPNRAPAGRVLLLNYIGGATNTGIVSKSASELVEAVDRDLRKMLINPSAVDPLVLGVRVWPQAIPQFLVGHLDLLEAAKSSLDRGGYDGLFLGGNYVAGVALGRCVEGAYESASQISDFLTKYAYK

SEQ ID NO: 15 - protein, 536 aa, Sorghum bicolor
MVAAAAMATAASAAAPLLNGTRRPARLRRRGLRVRCAAVAGGAAEAPASTGARLSADCVVVGGGISGLCTAQALATRHGVGEVLVTEARARPGGNITTVERPEEGYLWEEGPNSFQPSDPVLSMAVDSGLKDDLVFGDPNAPRFVLWEGKLRPVPSKPADLPFFDLMSIPGKLRAGLGALGIRPPPPGREESVEEFVRRNLGAEVFERLIEPFCSGVYAGDPSKLSMKAAFGKVWRLEEAGGSIIGGTIKTIQERGKNPKPPRDPRLPKPKGQTVASFRKGLAMLPNAITSSLGSKVKLSWKLTSITKSDGKGYVLEYETPEGVVLVQAKSVIMTIPSYVASDILRPLSGDAADALSRFYYPPVAAVTVSYPKEAIRKECLIDGELQGFGQLHPRSQGVETLGTIYSSSLFPNRAPAGRVLLLNYIGGATNTGIVSKTESELVEAVDRDLRKMLINSTAVDPLVLGVRVWPQAIPQFLVGHLDLLEVAKSALDQGGYDGLFLGGNYVAGVALGRCIEGAYESAAQIYDFLTKYAYK

SEQ ID NO: 16 - protein, 537 aa, Arabidopsis thaliana
MELSLLRPTTQSLLPSFSKPNLRLNVYKPLRLRCSVAGGPTVGSSKIEGGGGTTITTDCVIVGGGISGLCIAQALATKHPDAAPNLIVTEAKDRVGGNIITREENGFLWEEGPNSFQPSDPMLTMVVDSGLKDDLVLGDPTAPRFVLWNGKLRPVPSKLTDLPFFDLMSIGGKIRAGFGALGIRPSPPGREESVEEFVRRNLGDEVFERLIEPFCSGVYAGDPSKLSMKAAFGKVWKLEQNGGSIIGGTFKAIQERKNAPKAERDPRLPKPQGQTVGSFRKGLRMLPEAISARLGSKVKLSWKLSGITKLESGGYNLTYETPDGLVSVQSKSVVMTVPSHVASGLLRPLSESAANALSKLYYPPVAAVSISYPKEAIRTECLIDGELKGFGQLHPRTQGVETLGTIYSSSLFPNRAPPGRILLLNYIGGSTNTGILSKSEGELVEAVDRDLRKMLIKPNSTDPLKLGVRVWPQAIPQFLVGHFDILDTAKSSLTSSGYEGLFLGGNYVAGVALGRCVEGAYETAIEVNNFMSRYAYK

SEQ ID NO: 17 - protein, 548 aa, Nicotiana tabacum
MTTTPIANHPNIFTHQSSSSPLAFLNRTSFIPFSSISKRNSVNCNGWRTRCSVAKDYTVPSSAVDGGPAAELDCVIVGAGISGLCIAQVMSANYPNLMVTEARDRAGGNITTVERDGYLWEEGPNSFQPSDPMLTMAVDCGLKDDLVLGDPNAPRFVLWKGKLRPVPSKLTDLAFFDLMSIPGKLRAGFGAIGLRPSPPGHEESVEQFVRRNLGGEVFERLIEPFCSGVYAGDPSKLSMKAAFGKVWKLEETGGSIIGGTFKAIKERSSTPKAPRDPRLPKPKGQTVGSFRKGLRMLPDAISARLGSKLKLSWKLSSITKSEKGGYHLTYETPEGVVSLQSRSIVMTVPSYVASNILRPLSVAAADALSNFYYPPVGAVTISYPQEAIRDERLVDGELKGFGQLHPRTQGVETLGTIYSSSLFPNRAPKGRVLLLNYIGGAKNPEILSKTESQLVEVVDRDLRKMLIKPKAQDPLVVGVRVWPQAIPQFLVGHLDTLSTAKAAMNDNGLEGLFLGGNYVSGVALGRCVEGAYEVASEVTGFLSRYAYK

SEQ ID NO: 18 - protein, 511 aa, Eleusine indica
MAGSDDTRAAPARSVAVVGAGVSGLAAAYRLRKSGVNVTVFEAGDRAGGKIRSNSEGGFLWDEGANTMTESELEVSRLIDDLGLQDRQQYPNSQHKRYIVKDGAPVLIPSDPISLIKSSVLSTKSKFALFLEPFIYKKTSTRNSGIVSDEHLSESVGSFFERHFGQEVVDYLIDPFVAGTSGGDPESLSIRHAFPALWNLEKKYGSIIAGAILSKLTAKRDPVKKTSDSSGKRRNRRVSFSFLGGMQSLIDALHNEVGDGNVKLSTEVLSLACSVDGVPASGGWSISIDSKNTGSKEFGKKQTFDAVIMTAPLSNVQKMKFMKGGAPFVLDFLPKVDYLPLSLMVTAYKKEDVKRPLEGFGVLIPYKEQQKHGLKTLGTLFSSMMFPDRAPSDQFLYTTFIGGSHNRDLAGAPTTILKQLVTSDLRKLLGVEGQPTFVKHVYWRNAFPLYGRDYNSVLEAIEKMEKNLPGFFYAGNNKDGLAVGSVIASGSKAADLAISYLESRTKHDSSH

SEQ ID NO: 19 - protein, 511 aa, Eleusine indica; identical to SEQ ID NO: 3

SEQ ID NO: 20 - protein, 511 aa, Eleusine indica; identical to SEQ ID NO: 18

SEQ ID NO: 21 - protein, 533 aa, Amaranthus tuberculatus
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDCVLRAIDKMEKDLPGFFYAGNHKGGLSVGKAMASGCKAAELVISYLDSHIYVKMDEKTA

SEQ ID NO: 22 - protein, 534 aa, Amaranthus tuberculatus
MVIQSITHLSPNLALPSPLSVSTKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNILSAKSKLQIMLEPFLWRKHNATELSDEHVQESVGEFFERHFGKEFVDYVIDPFVAGTCGGDPQSLSMHHTFPEVWNIEKRFGSVFAGLIQSTLLSKKEKGGENASIKKPRVRGSFSFQGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLANASTDELKQIVSSDLQQLLGTEDEPSFVNHLFWSNAFPLYGHNYDSVLRAIDKMEKDLPGFFYAGNHKGGLSVGKAMASGCKAAELVISYLDSHIYVKMDEKTA

SEQ ID NO: 23 - protein, 535 aa, Amaranthus palmeri
MLIQSITHLSPKLALPSPLSISAKNYPVAVMGNISEREEPTSAKRVAVVGAGVSGLAAAYKLKSHGLSVTLFEADSRAGGKLKTVKKDGFIWDEGANTMTESEAEVSSLIDDLGLREKQQLPISQNKRYIARDGLPVLLPSNPAALLTSNFLSAKSKLQIMLEPFLWKKRNATELSDEHVQESVGEFFERHFGKEFVDYVIDPFVAGTCGGDPQSLSMHHTFPDVWNVEKRFGSVFAGLIQSTLLSKKEKGGGENASIKKPRVRGSFSFHGGMQTLVDTMCKQLGEDELKLQCEVLSLSYNQKGIPSLGNWSVSSMSNNTSEDQSYDAVVVTAPIRNVKEMKIMKFGNPFSLDFIPEVTYVPLSVMITAFKKDKVKRPLEGFGVLIPSKEQHNGLKTLGTLFSSMMFPDRAPSDMCLFTTFVGGSRNRKLAKASTDELKQIVSSDLQQLLGTEDEPSFVNHLFWSNAFPLYGHNYDSVLRAIDKMEKDLPGFFYAGNHKGGLSVGKAMASGCKAAELVISYLDSHLYVKMDEKTA

SEQ ID NO: 24 - protein, 544 aa, Sorghum bicolor
MLARTATVSSTSSHSHPYRPTSARSLRLRPVLAMAGSDDSRAAPARSVAVVGAGVSGLVAAYRLRKSGVNVTVFEAADRAGGKIRTNSEGGFLWDEGANTMTEGELEASRLIDDLGLQDKQQYPNSQHKRYIVKDGAPALIPSDPISLMKSSVLSTKSKIALFFEPFLYKKANTRNPGKVSDEHLSESVGSFFERHFGREVVDYLIDPFVAGTSAGDPESLSICHAFPALWNLERKYGSVVVGAILSKLTAKGDPVKTRRDSSAKRRNRRVSFSFHGGMQSLINALHNEVGDDNVKLGTEVLSLACTLDGAPAPGGWSISDDSKDASGKDLAKNQTFDAVIMTAPLSNVQRMKFTKGGAPFVLDFLPKVDYLPLSLMVTAFKKEDVKKPLEGFGVLIPYKEQQKHGLKTLGTLFSSMMFPDRAPDDQYLYTTFVGGSHNRDLAGAPTSILKQLVTSDLKKLLGVQGQPTFVKHIYWGNAFPLYGHDYNSVLEAIEKMEKNLPGFFYAGNNKDGLAVGSVIASGSKAADLAISYLESHTKHNNLH

SEQ ID NO: 25 - protein, 542 aa, Setaria italica
MLSSSTTTASPASSHPYRPAYPRASLRPVLAMAGSDDPRAAPARSVAVIGAGVSGLAAAYRLRKSGVNVTVFEAADRAGGKIRTNSEAGFLWDEGANTMTEGELEVSRLIDDLGLQDRQQYPNSQHKRYIVKDGAPALIPADPISLMKSSVLSTKSKLALFLEPFLYKKSNTRNSGKVSDEHLSESVGSFFERHFGREVVDYLIDPFVAGTSAGDPESLSIRHAFPALWNLERKYHSIIVGAILSKLTAKGDPVKTGSDLSGKRRNRRASFSFHGGMQSLINALHNEVGDDNVKLGTEVLSLACTFDGLPSTGGWSISVDSKDAGSKDLAKNQTFDAVIMTAPLSNVQRMKFRKGGAPFVLDFLPKVNYLPLSLMVTAFKKEDVKKPLEGFGVLIPYKEQQKHGLKTLGTLFSSMMFPDRAPDDQYLYTTFVGGSHNRDLAGAPTSILKQLVTSDLKKLLGVEGQPTFVKHIYWRNAFPLYGRDYGSVLDAIEKMEKNLPGFFYAGNNKDGLAVGNVIASGSKAAELAISYLESQTKHNNSH

SEQ ID NO: 26 - protein, 507 aa, Solanum tuberosum
MVPMAPSAGEDKQNCPKRVAVIGAGVSGLAAAYKLKIHGLDVTVFEAEGRAGGKLRSLSQDGLIWDEGANTMTESEGDVTFLLDSLGLREKQQFPLSQNKRYIARNGTPTLIPSNPIDLIKSNFLSTGSKLQMLFEPLLWKNKKLTKVADEHESVSGFFQRHFGKEVVDYLIDPFVAGTCGGDPDSLSMHLSFPELWNLEKRFGSVIVGAIRSKLSPIKEKKQGPPKTSVNKKRQRGSFSFLGGMQTLTDAICNDLKEDELRLNSRVLELSCSCSGDSATDSWSIFSASPHKRQAEEESFDAVIMTAPLCDVKGMKIAKRGNPFLLNFIPEVDYVPLSVVITTFKKESVKHPLEGFGVLVPSEEQKHGLKTLGTLFSSMMFPDRAPNNVYLYTTFVGGSRNRELAKASRTELKEIVTSDLKQLLGAEGEPTYVNHVCWSKAFPLYGHNYDSVLDAIDKMEKNLPGLFYAGNHKGGLSVGKALSSGCNAADLVISYLEAVSTDTKNHS

SEQ ID NO: 27 - DNA, 65 bp, Artificial
  Synthetically generated oligonucleotide
gcagccttcatactgagttttgaaggatcaccagcatagacaccttatgttaacattgaaactgt

SEQ ID NO: 28 - DNA, 53 bp, Artificial
  Synthetically generated oligonucleotide
gcagccttcatactgagttttgaaggatcaccagtatagacaccttatgttaa

SEQ ID NO: 29 - DNA, 39 bp, Artificial
  Synthetically generated oligonucleotide
gcagccttcatactgagttttgaaggatcaccagtatag

SEQ ID NO: 30 - DNA, 39 bp, Artificial; identical to SEQ ID NO: 29
  Synthetically generated oligonucleotide

SEQ ID NO: 31 - DNA, 39 bp, Artificial; identical to SEQ ID NO: 29
  Synthetically generated oligonucleotide

SEQ ID NO: 32 - DNA, 38 bp, Artificial
  Synthetically generated oligonucleotide
gcagccttcatactgagttttgaaggatcaccagtata

SEQ ID NO: 33 - DNA, 41 bp, Artificial
  Synthetically generated oligonucleotide
gcagccttcatactgagttttgaaggatcaccagtatagac

SEQ ID NO: 34 - DNA, 41 bp, Artificial; identical to SEQ ID NO: 33
  Synthetically generated oligonucleotide

SEQ ID NO: 35 - DNA, 41 bp, Artificial; identical to SEQ ID NO: 33
  Synthetically generated oligonucleotide

SEQ ID NO: 36 - DNA, 42 bp, Artificial
  Synthetically generated oligonucleotide
gcagccttcatactgagttttgaaggatcaccagtatagaca

SEQ ID NO: 37 - DNA, 42 bp, Artificial; identical to SEQ ID NO: 36
  Synthetically generated oligonucleotide

SEQ ID NO: 38 - DNA, 42 bp, Artificial; identical to SEQ ID NO: 36
  Synthetically generated oligonucleotide

SEQ ID NO: 39 - DNA, 42 bp, Artificial; identical to SEQ ID NO: 36
  Synthetically generated oligonucleotide

SEQ ID NO: 40 - DNA, 45 bp, Artificial
  Synthetically generated oligonucleotide
gcagccttcatactgagttttgaaggatcaccagtatagacacct

SEQ ID NO: 41 - DNA, 42 bp, Artificial; identical to SEQ ID NO: 36
  Synthetically generated oligonucleotide

SEQ ID NO: 42 - DNA, 42 bp, Artificial; identical to SEQ ID NO: 36
  Synthetically generated oligonucleotide

SEQ ID NO: 43 - DNA, 46 bp, Artificial
  Synthetically generated oligonucleotide
gcagccttcatactgagttttgaaggatcaccagtatagacacccg

SEQ ID NO: 44 - DNA, 46 bp, Artificial
  Synthetically generated oligonucleotide
gcagccttcatactgagttttgaaggatcaccagtatagacacctg

SEQ ID NO: 45 - DNA, 46 bp, Artificial; identical to SEQ ID NO: 44
  Synthetically generated oligonucleotide

SEQ ID NO: 46 - DNA, 46 bp, Artificial; identical to SEQ ID NO: 44
  Synthetically generated oligonucleotide

SEQ ID NO: 47 - DNA, 44 bp, Artificial
  Synthetically generated oligonucleotide
gcagccttcatactgagttttgaaggatcaccagtatagacacc

SEQ ID NO: 48 - DNA, 46 bp, Artificial; identical to SEQ ID NO: 44
  Synthetically generated oligonucleotide

SEQ ID NO: 49 - DNA, 48 bp, Artificial
  Synthetically generated oligonucleotide
gcagccttcatactgagttttgaaggatcaccagtatagacacctgag

SEQ ID NO: 50 - DNA, 48 bp, Artificial; identical to SEQ ID NO: 49
  Synthetically generated oligonucleotide

SEQ ID NO: 51 - DNA, 48 bp, Artificial; identical to SEQ ID NO: 49
  Synthetically generated oligonucleotide

SEQ ID NO: 52 - DNA, 48 bp, Artificial; identical to SEQ ID NO: 49
  Synthetically generated oligonucleotide

SEQ ID NO: 53 - DNA, 47 bp, Artificial
  Synthetically generated oligonucleotide
gcagccttcatactgagttttgaaggatcaccagtatagacacctga

SEQ ID NO: 54 - DNA, 49 bp, Artificial
  Synthetically generated oligonucleotide
gcagccttcatactgagttttgaaggatcaccagtatagacacctgagc

SEQ ID NO: 55 - DNA, 49 bp, Artificial; identical to SEQ ID NO: 54
  Synthetically generated oligonucleotide

SEQ ID NO: 56 - DNA, 50 bp, Artificial
  Synthetically generated oligonucleotide
gcagccttcatactgagttttgaaggatcaccagtatagacacctgagca

SEQ ID NO: 57 - DNA, 50 bp, Artificial; identical to SEQ ID NO: 56
  Synthetically generated oligonucleotide

SEQ ID NO: 58 - DNA, 40 bp, Artificial
  Synthetically generated oligonucleotide
tgctggtgatccttcaaaactcagtatgaaggctgcattt

SEQ ID NO: 59 - protein, 13 aa, Artificial
  Synthetically generated peptide
AGDPSKLSMKAAF

SEQ ID NO: 60 - DNA, 40 bp, Artificial
  Synthetically generated oligonucleotide
tactggtgatccttcaaaactcagtatgaaggctgcattt

SEQ ID NO: 61 - protein, 13 aa, Artificial
  Synthetically generated peptide
TGDPSKLSMKAAF

SEQ ID NO: 62 - DNA, 40 bp, Artificial; identical to SEQ ID NO: 60
  Synthetically generated oligonucleotide

SEQ ID NO: 63 - protein, 13 aa, Artificial
  Synthetically generated peptide
TGDPSKLSMKAAF

SEQ ID NO: 64 - protein, 59 aa, Eleusine indica
LIEPFCSGVYAGDPSKLSMKAAFGKVWRLEEAGGSIIGGTIKTIQERGKNPKPERDPRL

SEQ ID NO: 65 - protein, 59 aa, Eleusine indica
LIEPFCSGVYTGDPSKLSMKAAFGKVWRLEEAGGSIIGGTIKTIQERGKNPKPERDPRL

SEQ ID NO: 66 - protein, 59 aa, Eleusine indica; identical to SEQ ID NO: 65

SEQ ID NO: 67 - protein, 59 aa, Arabidopsis thaliana
LIEPFCSGVYAGDPSKLSMKAAFGKVWKLEQNGGSIIGGTFKAIQERKNAPKAERDPRL

SEQ ID NO: 68 - protein, 59 aa, Nicotiana tabacum
LIEPFCSGVYAGDPSKLSMKAAFGKVWKLEETGGSIIGGTFKAIKERSSTPKAPRDPRL

SEQ ID NO: 69 - protein, 59 aa, Sorghum bicolor
LIEPFCSGVYAGDPSKLSMKAAFGKVWRLEEAGGSIIGGTIKTIQERGKNPKPPRDPRL

SEQ ID NO: 70 - protein, 59 aa, Setaria italica; identical to SEQ ID NO: 69

SEQ ID NO: 71 - protein, 59 aa, Oryza sativa
LIEPFCSGVYAGDPSKLSMKAAFGKVWRLEDTGGSIIGGTIKTIQERGKNPKPPRDPRL

SEQ ID NO: 72 - protein, 59 aa, Brachypodium distachyon
LIEPFCSGVYAGDPSKLSMRAAFGKVWRLEEIGGSIIGGTIKAIQDRGKNPKPPRDPRL

SEQ ID NO: 73 - protein, 60 aa, Sorghum bicolor
LIDPFVAGTSAGDPESLSICHAFPALWNLERKYGSVVVGAILSKLTAKGDPVKTRRDSSA

SEQ ID NO: 74 - protein, 60 aa, Zea mays
FVDPFVAGTSAGDPESLSIRHAFPALWNLERKYGSVIVGAILSKLAAKGDPVKTRHDSSG

SEQ ID NO: 75 - protein, 60 aa, Setaria italica
LIDPFVAGTSAGDPESLSIRHAFPALWNLERKYHSIIVGAILSKLTAKGDPVKTGSDLSG

SEQ ID NO: 76 - protein, 60 aa, Eleusine indica
LIDPFVAGTSGGDPESLSIRHAFPALWNLEKKYGSVIAGAILSKLTAKRDPVKKTSDSSG

SEQ ID NO: 77 - protein, 60 aa, Eleusine indica; identical to SEQ ID NO: 76

SEQ ID NO: 78 - protein, 60 aa, Eleusine indica
LIDPFVAGTSGGDPESLSIRHAFPALWNLEKKYGSIIAGAILSKLTAKRDPVKKTSDSSG

SEQ ID NO: 79 - protein, 59 aa, Amaranthus palmeri
VIDPFVAGTCGGDPQSLSMHHTFPDVWNVEKRFGSVFAGLIQSTLLSKKEKGGGENASI

SEQ ID NO: 80 - protein, 57 aa, Amaranthus tuberculatus
VIDPFVAGTCGDPQSLSMHHTFPEVWNIEKRFGSVFAGLIQSTLLSKKEKGGENASI

SEQ ID NO: 81 - protein, 60 aa, Solanum tuberosum
LIDPFVAGTCGGDPDSLSMHLSFPELWNLEKRFGSVIVGAIRSKLSPIKEKKQGPPKTSV

SEQ ID NO: 82 - protein, 60 aa, Glycine max
LIDPFVGGTSAADPESLSMRHSFPELWNLEKRFGSIIAGALQSKLFAKREKTGENRTALR

SEQ ID NO: 83 - protein, 60 aa, Arabidopsis thaliana
LIDPFVGGTSAADPDSLSMKHSFPDLWNVEKSFGSIIVGAIRTKFAAKGGKSRDTKSSPG

SEQ ID NO: 84 - protein, 60 aa, Nicotiana tabacum
LIDPFVAGTCGGDPDSLSMHHSFPELWNLEKRFGSVILGAIRSKLSPKNEKKQGPPKTSA

SEQ ID NO: 85 - protein, 502 aa, Glycine max
MASSATDDNPRSVKRVAVVGAGVSGLAAAYKLKSHGLDVTVFEAEGRAGGRLRSVSQDGLIWDEGANTMTESEIEVKGLIDALGLQEKQQFPISQHKRYIVKNGAPLLVPTNPAALLKSKLLSAQSKIHLIFEPFMWKRSDPSNVCDENSVESVGRFFERHFGKEVVDYLIDPFVGGTSAADPESLSMRHSFPELWNLEKRFGSIIAGALQSKLFAKREKTGENRTALRKNKHKRGSFSFQGGMQTLTDTLCKELGKDDLKLNEKVLTLAYGHDGSSSSQNWSITSASNQSTQDVDAVIMTAPLYNVKDIKITKRGTPFPLNFLPEVSYVPISVMITTFKKENVKRPLEGFGVLVPSKEQKNGLKTLGTLFSSMMFPDRAPSDLYLYTTFIGGTQNRELAQASTDELRKIVTSDLRKLLGAEGEPTFVNHFYWSKGFPLYGRNYGSVLQAIDKIEKDLPGFFFAGNYKGGLSVGKAIASGCKAADLVISYLNSASDNTVPDK

SEQ ID NO: 86 - protein, 478 aa, Arabidopsis thaliana
MASGAVADHQIEAVSGKRVAVVGAGVSGLAAAYKLKSRGLNVTVFEADGRVGGKLRSVMQNGLIWDEGANTMTEAEPEVGSLLDDLGLREKQQFPISQKKRYIVRNGVPVMKKSSKVSDASAEESVSEFFQRHFGQEVVDYLIDPFVGGTSAADPDSLSMKHSFPDLWNVEKSFGSIIVGAIRTKFAAKGGKSRDTKSSPGTKKGSRGSFSFKGGMQILPDTLCKSLSHDEINLDSKVLSLSYNSGSRQENWSLSCVSHNETQRQNPHYDAVIMTAPLCNVKEMKVMKGGQPFQLNFLPEINYMPLSVLITTFTKEKVKRPLEGFGVLIPSKEQKHGFKTLGTLFSSMMFPDRSPSDVHLYTTFIGGSRNQELAKASTDELKQVVTSDLQRLLGVEGEPVSVNHYYWRKAFPLYDSSYDSVMEAIDKMENDLPGFFYAGNHRGGLSVGKSIASGCKAADLVISYLESCSNDKKPNDSL

SEQ ID NO: 87 - protein, 504 aa, Nicotiana tabacum
MAPSAGEDKHSSAKRVAVIGAGVSGLAAAYKLKIHGLNVTVFEAEGKAGGKLRSVSQDGLIWDEGANTMTESEGDVTFLIDSLGLREKQQFPLSQNKRYIARNGTPVLLPSNPIDLIKSNFLSTGSKLQMLLEPILWKNKKLSQVSDSHESVSGFFQRHFGKEVVDYLIDPFVAGTCGGDPDSLSMHHSFPELWNLEKRFGSVILGAIRSKLSPKNEKKQGPPKTSANKKRQRGSFSFLGGMQTLTDAICKDLREDELRLNSRVLELSCSCTEDSAIDSWSIISASPHKRQSEEESFDAVIMTAPLCDVKSMKIAKRGNPFLLNFIPEVDYVPLSVVITTFKRENVKYPLEGFGVLVPSKEQQHGLKTLGTLFSSMMFPDRAPNNVYLYTTFVGGSRNRELAKASRTELKEIVTSDLKQLLGAEGEPTYVNHLYWSKAFPLYGHNYDSVLDAIDKMEKNLPGLFYAGNHRGGLSVGKALSSGCNAADLVISYLESVSTDSKRHC

(19) United States
(12) Patent Application Publication
(10) Pub. No.: US 2022/0282271 A1
(43) Pub. Date: Sep. 8, 2022

(54) HERBICIDE RESISTANT PLANTS AND METHODS OF MAKING AND USING

(71) Applicants: OFFICE OF INNOVATION ADVANCEMENT AND COMMERCIALIZATION, Auburn, AL (US); NUTECH VENTURES, Lincoln, NE (US)

(72) Inventors: Joseph S. McElroy, Auburn, AL (US); Thomas E. Clemente, Lincoln, NE (US)

(21) Appl. No.: 17/763,134

(22) PCT Filed: Sep. 23, 2020
(86) PCT No.: PCT/US2020/052292

§ 371 Date: Mar. 23, 2022

Related U.S. Application Data
(60) Provisional application No. 62/904,270, filed on Sep. 23, 2019.

(51) Int. Cl. C12N 15/82; C12N 9/02

(52) U.S. Cl. CPC C12N 15/8274; C12N 9/001; C12Y 103/03004

(57) ABSTRACT

The present disclosure relates to protoporphyrinogen IX oxidase (PPO) polypeptides, and nucleic acids encoding such polypeptides. The present disclosure describes a mutation in a PPO sequence that imparts herbicide-resistance, particularly oxadiazole-resistance, to plants.

### CROSS REFERENCE TO RELATED APPLICATIONS

This application claims the benefit of priority under 35 U.S.C. § 119(e) to U.S. Application No. 62/904,270 filed on Sep. 23, 2019.

### TECHNICAL FIELD

This disclosure generally relates to herbicide resistance in plants.

### BACKGROUND

Weeds are a constant problem in farm fields. Weeds not only compete with crops for water, nutrients, sunlight, and space, but also harbor insects and diseases, clog irrigation and drainage systems, undermine crop quality, and deposit weed seeds into crop harvests. If left uncontrolled, weeds can reduce crop yields significantly. Farmers can fight weeds with tillage, hand weeding, herbicides, or combinations thereof.

Broad-spectrum or non-selective herbicides can be applied to a field to reduce weed growth just before the crop germinates to prevent the crops from being killed together with the weeds. Weeds that emerge during the growing season can be controlled using narrow-spectrum or selective herbicides. However, due to the presence of different types of weeds that emerge, this method can be costly and can harm the environment.

Herbicide resistant crops provide farmers a vital tool in fighting weeds. Herbicide resistant crops give farmers the flexibility to apply herbicides only when needed, to control total input of herbicides and to use herbicides with preferred environmental characteristics.

### SUMMARY

Described herein are protoporphyrinogen IX oxidase (PPO) polypeptides, and nucleic acids encoding such polypeptides. Also described herein is a mutation in a PPO sequence that imparts herbicide-resistance, particularly oxadiazole-resistance, to plants.

The novel mutation described herein that confers oxadiazole-resistance is the first evidence of a direct role of PPO1 in PPO-resistance and the first evidence of evolved resistance in PPO1.

In one aspect, plants having a mutation in a gene encoding a polypeptide having protoporphyrinogen IX oxidase activity are provided, where the mutation includes a substitution of an alanine (A) to a threonine (T) at residue 212 (relative to SEQ ID NO:1 when aligned using BLAST) and imparts a phenotype of herbicide resistance to the plant. As described herein, the herbicide is an oxadiazole.

In some embodiments, the plant is selected from wheat, corn, soybean, tobacco, brachiaria, rice, millet, barley, tomato, apple, pear, strawberry, orange, alfalfa, cotton, carrot, potato, sugar beets, yam, lettuce, spinach, petunia, rose, chrysanthemum, turf grass, pine, fir, spruce, heavy metal accumulating plants, sunflower, safflower, rapeseed, and Arabidopsis.

In some embodiments, the mutation is a point mutation. In some embodiments, the herbicide is oxadiazon.

In one aspect, seed produced from such a plant is provided. In another aspect, progeny of such a plant is provided.

In another aspect, methods of making a herbicide-resistant plant are provided. Such methods typically include the steps of: a) mutagenizing plant cells; b) obtaining one or more plants from the cells; and c) identifying at least one of the plants that contains a mutation in a gene encoding a polypeptide having a wild-type sequence as shown in SEQ ID NO:1 and exhibiting protoporphyrinogen IX oxidase activity. As described herein, the mutation includes a substitution of an alanine (A) to a threonine (T) at residue 212 (relative to SEQ ID NO:1 when aligned using BLAST) and imparts a phenotype of herbicide resistance to the plant. As described herein, the herbicide is an oxadiazole.

In some embodiments, the mutagenizing utilizes a chemical mutagen, ionizing radiation, or fast neutron bombardment. In some embodiments, the mutagenizing step comprises CRISPR, TALEN, or zinc-finger nuclease.

In some embodiments, the plant cells are selected from wheat, corn, soybean, tobacco, brachiaria, rice, millet, barley, tomato, apple, pear, strawberry, orange, alfalfa, cotton, carrot, potato, sugar beets, yam, lettuce, spinach, petunia, rose, chrysanthemum, turf grass, sunflower, safflower, rapeseed, and Arabidopsis. In some embodiments, the plant cells are in a seed.

In some embodiments, the mutagenizing step is performed on seed from the plant. In some embodiments, the mutation is a point mutation. In some embodiments, the herbicide is oxadiazon.

In another aspect, methods for producing a plant are provided. Such methods typically include the steps of: a) providing a first plant and a second plant, where the first plant has a mutation in an endogenous gene encoding a polypeptide having a wild-type sequence as shown in SEQ ID NO:1 and exhibits protoporphyrinogen IX oxidase activity, where the mutation includes a substitution of an alanine (A) to a threonine (T) at residue 212 (relative to SEQ ID NO:1 when aligned using BLAST) and imparts a phenotype of herbicide resistance to the plant, and where the herbicide is an oxadiazole; and where the second plant exhibits a desired phenotypic trait; b) crossing the first plant with the second plant to produce one or more F1 progeny plants; c) collecting seed produced by the F1 progeny plants; and d) germinating the seed to produce plants having a phenotype of herbicide resistance.

In some embodiments, the second plant contains a desired phenotypic trait selected from the group consisting of disease resistance; high yield; high grade index; curability; curing quality; mechanical harvestability; holding ability; leaf quality; height; maturation; stalk size; and leaf number per plant.

In some embodiments, such methods further include the steps of: crossing the at least one of the plants that contains the mutation with a second plant; and selecting progeny of the cross that have the at least one mutation, wherein the plant is homozygous for the at least one mutation.

In still another aspect, methods for producing a protoporphyrinogen IX oxidase (PPO) mutant plant are provided. Such methods typically include a) providing at least one nucleic acid to a plant cell, where the nucleic acid comprises a guide RNA, a nucleic acid modification template comprising at least one nucleic acid modification of the PPO nucleic acid sequence, and an endonuclease, where the guide RNA and the endonuclease are capable of forming a complex that enables the endonuclease to introduce a double strand break at a target site in the genome of the plant cell, and where the at least one nucleotide modification comprises a substitution of an alanine (A) to a threonine (T) at residue 212 (relative to SEQ ID NO:1 when aligned using BLAST); b) obtaining a plant from the plant cell of (a); c) evaluating the plant of (b) for the presence of the at least one nucleotide modification; and, d) selecting a progeny plant that shows resistance to oxadiazole.

In some embodiments, the endonuclease is a Cas endonuclease or Cpf1 endonuclease. In some embodiments, the plant cell is a protoplast.

In yet another aspect, a nucleic acid operably linked to a heterologous promoter is provided, where the nucleic acid encodes a protoporphyrinogen IX oxidase (PPO) having a threonine at position 212 (relative to SEQ ID NO:1 when aligned using BLAST). In some embodiments, the nucleic acid has at least about 50% sequence identity (e.g., at least about 60%, 70%, 75%, 80%, 85%, 90%, 95%, 96%, 97%, 98%, 99% or 100%) to SEQ ID NO:2.

In one aspect, a vector is provided that includes such a nucleic acid. In some embodiments, the vector is a plant transformation vector.

In another aspect, host cells that contain such nucleic acids are provided. In some embodiments, the host cell is a bacterial cell or a plant cell.

In another embodiment, transgenic plants are provided that are transformed with a nucleic acid molecule encoding a PPO polypeptide that includes an A212T substitution and imparts a phenotype of herbicide resistance to the plant. As described herein, the herbicide is an oxadiazole.

In still another embodiment, oxadiazole-resistant plant seeds are provided. Typically, the seed includes a chimeric plant gene having: i) a promoter functional in plant cells; ii) a nucleic acid sequence encoding a chloroplast transit peptide; iii) a nucleic acid sequence encoding a PPO polypeptide comprising a threonine at position 212 (relative to SEQ ID NO:1 when aligned using BLAST). Typically the promoter is heterologous with respect to the nucleic acid sequence encoding the PPO polypeptide and allows sufficient expression of the PPO polypeptide to increase the oxadiazole resistance of a plant produced from the seed.

In some embodiments, the promoter is the CaMV35S promoter.

In one aspect, seed produced from such plants or progeny of such plants are provided.

In one aspect, methods of making an oxadiazole-resistant plant are provided.

Such methods typically include (a) introducing a nucleic acid into a plurality of plant cells to produce transformed plant cells, wherein the nucleic acid encodes a PPO polypeptide comprising a threonine at position 212 (relative to SEQ ID NO:1 when aligned using BLAST); (b) selecting at least one oxadiazole-resistant plant cell from the transformed plant cells; and (c) regenerating an oxadiazole-resistant plant from the at least one oxadiazole-resistant plant cell selected in step (b); such that, when the plant is exposed to oxadiazole, the plant is resistant to the oxadiazole.

In some embodiments, the plant cell is a protoplast. In some embodiments, the plant cells are produced from a tissue type selected from the group consisting of leaves, pollen, embryos, cotyledons, hypocotyls, meristematic cells, roots, root tips, anthers, flowers, stems and pods.

In another aspect, methods for selectively controlling weeds in a field containing a crop plant are provided. Such methods typically include applying a sufficient amount of oxadiazole to a field in which a crop plant as described herein is growing to control the weeds without significantly affecting the crop plant.

In some embodiments, a sufficient amount of oxadiazole is an amount that provides at least 50% (e.g., at least 60%, 70%, 75%, 80%, 85%, 90%, 95%, 99% or 100%) control of a weed species in the field.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 are graphs showing the percent injury response relative to the nontreated control of three Eleusine indica biotypes 14 DAT with increasing rates of four different protoporphyrinogen oxidase (PPO) inhibitors: oxadiazon, sulfentrazone, flumioxazin, lactofen. Response was modeled based on the log rate of the herbicides to create equal spacing between rates using sigmoidal regression of percent injury relative to the nontreated control. Non-log transformed herbicide rates are presented for reference. Means are represented by differing symbols for each biotype and regression equation models are represented by differing line type for each biotype. Vertical bars represent standard errors (P=0.05). E. indica biotype abbreviations: S, known susceptible wild-type; R1, oxadiazon resistant biotype from Country Club of Virginia, Richmond, Va.; R2, oxadiazon resistant biotype from River Bend Golf Course, New Bern, N.C.

FIG. 2A is a portion of a protein alignment of PPO1 among three Eleusine indica biotypes. The codon containing the single nucleotide polymorphism for the A212T mutation is depicted in the rectangle.

FIG. 2B is an alignment showing the amino acid sequence conservation of a portion of the PPO1 and PPO2 sequences in plant species. A conserved amino acid residue at position 212 in PPO1 is indicated by a rhombus. Abbreviations: PPO1, chloroplst-targeted protoporphyrinogen oxidase (PPO); PPO2, mitochondria targeted PPO. The sequences listed in Table 4 were used to establish the alignment.

FIG. 3A-3B are photographs showing protein expression of PPO1 alleles from three Eleusine indica biotypes transformed with the hemG mutant E. coli strain SASX38. FIG. 3A shows an E. coli strain grown on LB media alone, or supplemented with hematin (10 μg/mL), or in presence of oxadiazon (50 μM). FIG. 3B shows an E. coli strain grown on LB media containing increasing concentrations of oxadiazon (0, 10, 50, 100, 200 μM). E. coli isolates were as follows: NT, non-transformed E. coli strain SASX38; S, E. coli strain SASX38 transformed with a vector encoding S biotype PPO1 with Ala212; R1, E. coli strain SASX38 transformed with R1 PPO1 with A212T; R2, hemG mutant E. coli strain SASX38 transformed with R2 PPOL.

FIG. 4 is a schematic showing the position of the A212T mutation relative to the predicted binding mode of oxadiazon. Oxadiazon (cyan sticks) was modeled into the binding-site of the homology model (gray with secondary structure elements in cartoon style) of Eleusine indica PPO1 protein. Alanine 212 (green sticks) is present in the wild type model, threonine 212 (magenta sticks) is present in A212T mutation model. The hydroxyl-group of Thr212 can form an intramolecular hydrogen bond to the carbonyl backbone of tyrosine 211. As a result, it creates close several steric contacts (2.75 Å-3.19 Å depicted as orange dashed lines) with the modeled ligand oxadiazon. The FAD cofactor (gray) is partially visible. Other amino acid side chains are not shown to improve clarity.

FIG. 5 is a photograph of tobacco seed carrying the E. indica PPO transgenic allele germinating on medium supplemented (left to right) with 0 mg/l, 0.5 mg/l, 1.0 mg/l, 3 mg/l, and 5 mg/l oxadiazon, respectively. Panel A, wild type control tobacco seeds; Panel B, transgenic event 32 Xanthi seeds; Panel C, transgenic event 33 tobacco seeds. Image taken 13 days after plating.

FIG. 6 is a graph showing transformed plants containing the A212T amino acid substitution in PPO1 (T22, T26, T32, T33, and T38) exposed to oxadiazon (0.25, 0.50, 1.0, 2.0, and 4.0 lb ai/a), lactofen (0.19 lb ai/a), glyphosate (0.5 lb ai/a), and a non-treated control 3 DAT (FIG. 6A) and 7 DAT (FIG. 6B).

FIG. 7 is a protein alignment with PPO1 and PPO2 from a number of different species. The protein structure on the bottom is modeled as the template of mitochondrial PPO2 in Nicotiana tabacum (mtPPO2, PDB entry: 1SEZ). The ‘right arrow’ shows the α-helix secondary structure, the ‘rectangle’ shows the β-sheet secondary structure. The red color shows the FAD binding domain, the green color shows the substrate-binding domain, the blue color shows the membrane-binding domain. The codon containing PPO1 A212T substitution is indicated by a blue rhombus. Abbreviations: S, known susceptible E. indica wild-type (SEQ ID NO:10); R1, oxadiazon resistant E. indica biotype from Country Club of Virginia, Richmond, Va. (SEQ ID NO:11); R2, oxadiazon resistant E. indica biotype from River Bend Golf Course, New Bern, N.C. (SEQ ID NO:12). PPO1, chloroplast-targeted PPO1; PPO2, mitochondrial-targeted PPO2. In addition to the above sequences, the following sequences were used to establish the alignment: PPO1: Arabidopsis thaliana (At; NP_192078 (SEQ ID NO:13); N. tabacum (Nt; BAA34713 (SEQ ID NO:14)); Setaria italica (Si; XP_004967639 (SEQ ID NO:15)); Sorghum bicolor (Sb; XP_002455484 (SEQ ID NO: 16)); Amaranthus tuberculatus (AmT; ABD52324 (SEQ ID NO:17)). PPO2: At (NP_001190307 (SEQ ID NO:18)); PPO inhibitor resistant A. tuberculatus (AmT_r; ABD52328 (SEQ ID NO:19)); PPO inhibitor susceptible A. tuberculatus (AmT_s; ABD52326 (SEQ ID NO:20)); Si (XP_004976030 (SEQ ID NO:21)); Sb (XP 002446710 (SEQ ID NO:22)); Amaranthus palmeri (Ap; ATE88443 (SEQ ID NO:23)); Solanum tuberosum (St; XP_006356026 (SEQ ID NO:24)); Glycine max (Gm; NP_001236376 (SEQ ID NO:25)); Nt (NP 001312887 (SEQ ID NO:26)).

FIG. 8A-8B show reads mapping of Eleusine indica PPO1 and PPO2 gene referenced with genome DNA scaffold. FIG. 8A shows reads extraction of chloroplast-targeted PPO1. The annotated bar shows the exons numbers and locations. FIG. 8B shows reads extraction of mitochondrial-targeted PPO2. The annotated bar shows the introns numbers and locations.

FIG. 9 shows reads mapping to the mutation site PPO1 A212T in the R1 Eleusine indica biotype referenced with genome DNA scaffold. The reads showed the nucleotide in transcriptome reads at position 634 is thymine, while in genomic DNA is cytosine. When translated to amino acids, the R1 E. indica biotype has a threonine at position 212, while the S E. indica biotype has an alanine at position 212. Top to bottom, SEQ ID NO:27-SEQ ID NO:57.

FIG. 10A-10F are graphs showing the percent effects of protoporphyrinogen oxidase (PPO) inhibitors on in vitro enzyme activity of Eleusine indica wild-type PPO1 and PPO1 A212T variants. Six different PPO inhibitors belonging to five different structurally unrelated chemical families were used. The unit of dose rate is mole (M). FIG. 10A.1 shows the IC50 of wild-type E. indica PPO1 for oxadiazon, while there were no IC50 results obtained for variant E. indica PPO1 A212T because it is completely resistant. FIG. 10B.1 shows the IC50 of wild-type E. indica PPO1 for sulfentrazone. FIG. 10B.2 shows the IC50 of variant E. indica PPO1 A212T for sulfentrazone. FIG. 10C.1 shows the IC50 of wild-type E. indica PPO1 for saflufenacil. FIG. 10C.2 shows the IC50 of variant E. indica PPO1 A212T for saflufenacil. FIG. 10D.1 shows the IC50 of wild-type E. indica PPO1 for lactofen. FIG. 10D.2 shows the IC50 of variant E. indica PPO1 A212T for lactofen. FIG. 10E.1 shows the IC50 of wild-type E. indica PPO1 for flumioxazin. FIG. 10E.2 shows the IC50 of variant E. indica PPO1 A212T for flumioxazin. FIG. 10F.1 shows the IC50 of wild-type E. indica PPO1 for trifludimoxazin. FIG. 10F.2 shows the IC50 of variant E. indica PPO1 A212T for trifludimoxazin.

FIG. 11A is a schematic that shows the structure of the plant transformation vector introduced into soybean.

FIG. 11B is a photograph of transgenic soybean plants selected first for glufosinate. A second selection is performed with oxadizaon.

### DETAILED DESCRIPTION

Protoporphyrinogen IX oxidase (PPO or protox) (EC 1.3.3.4) is an oxygen-dependent enzyme that catalyzes a step in the biosynthesis of chlorophyll and heme, catalyzing the oxidation of protoporphyrinogen IX to protoporphyrin IX. PPO has two isoforms, PPO1 and PPO2, which are encoded by two nuclear genes, PPO1 and PPO2. PPO1 is located in the envelope membranes of chloroplasts, and PPO2 is located on the outer surface of the inner mitochondrial membrane. In some prokaryotes and plant species, PPO2 can dual-target to both chloroplast and mitochondria.

Mitochondrial-targeted PPO2 from Nicotiana tabacum (mtPPO2) has three domains: a FAD-binding domain, a membrane-binding domain and a substrate-binding domain. The homology similar amino acid sequence indicates that the crystal structure of PPO1 would resemble the structure of PPO2 in higher plants. The sequence of the PPO1 nucleic acid from Eleusine indica (L.) Gaertn (goosegrass) is shown in SEQ ID NO:2 and the encoded polypeptide sequence is shown in SEQ ID NO:1; the sequence of the PPO2 nucleic acid from Eleusine indica (L.) Gaertn (goosegrass) is shown in SEQ ID NO:4 and the encoded polypeptide sequence is shown in SEQ ID NO:3. Any number of endogenous or exogenous PPO sequences can be used, however, in the methods described herein. Simply by way of example, PPO sequences can be found in GenBank Accession Nos. NP_001236376.1 (GI: 351726950) from Glycine max; AAG00946.1 (GI 9857979) from Zea mays; NP_192078 or NP_001190307 from Arabidopsis thaliana; BAA34713 or NP_001312887 from Nicotiana tabacum; XP 004967639 or XP_004976030 from Setaria italica; XP_002455484 or XP_002446710 from Sorghum bicolor; ABD52324, ABD52328, or ABD52326 from Amaranthus tuberculatus; ATE88443 from Amaranthus palmeri; XP_006356026 from Solanum tuberosum.

PPO1 and PPO2 are herbicide targets of a number of herbicides (i.e., PPO-inhibiting herbicides). When PPO is inhibited, the substrate, protoporphyrinogen IX, accumulates and is exported into the cytoplasm, and the catalytic product of PPO, protoporphyrin IX, accumulates in the cytoplasm. Protoporphyrin IX induces the formation of singlet oxygen in the presence of light, causing lipid peroxidation and cell membrane leakage.

While PPO-inhibiting herbicides have been commercially available since the 1980s, resistance to these compounds has evolved relatively slowly. To date, there are only thirteen plant species with confirmed resistance to PPO inhibitors, compared to 48, 160, and 43 species for acetyl-CoA caroboxylase (ACCase) inhibitors, acetolactate synthase (ALS) inhibitors, and 5-enolpyruvyl shikimate 3-phospate (EPSP) synthase inhibitors, respectively. The low number of plant species resistant to PPO inhibitors is partially attributed to the presence of the two isoforms.

Thus, both PPO1 and PPO2 are targets for PPO inhibitors, even though they are located in different organelles. Interestingly, however, all the mutations identified to-date that confer resistance to PPO-inhibitors have been in the mitochondrial-targeted PPO2. In all cases, however, the reported PPO2 target-site mutations did not provide complete prophylaxis against injury but did allow for greater survival for individuals carrying the mutation. Prior to this disclosure, there have been no reported field-evolved resistant mutations in the chloroplast-targeted PPO1 that confer resistance to PPO-inhibitors in weed species.

Oxadiazon is a unique PPO inhibitor utilized for pre-emergence control of Eleusine indica. According to the Herbicide Resistance Action Committee (HRAC) and based upon their mode of action, PPO-inhibiting herbicides are classified in class E, which includes diphenyl ethers, phenylpyrazoles, triazolinones, thiadiazoles, oxadiazoles, pyrimidinediones, oxazolidinedione, and N-phenylphthalimides, all of which are structurally unrelated herbicide chemical families.

Two E. indica biotypes were previously shown to be resistant to oxadiazon but not to other structurally unrelated PPO inhibitors such as lactofen, flumioxazin and sulfentrazone. Using the two oxadiazon-resistant E. indica biotypes, a novel mutation, A212T, has been identified in the chloroplast-targeted PPO1 that, as described herein, confers resistance to oxadiazon in a heterologous expression system and in transgenic plants. Computational structural modeling indicated that the presence of a methyl group on the threonine at position 212 changes the PPO1 active site and produces repulsive electrostatic interactions that repel oxadiazon from the binding pocket.

The novel mutation described herein in PPO1 confers specific resistance to the PPO inhibitor, oxadiazon, while causing no cross-resistance to a number of other herbicides evaluated. While not wishing to be bound by any particular theory, it appears that the mutation described herein inhibits herbicides through conjugate exclusion, despite being located within the catalytic domain. Thus, an oxadiazole herbicide (e.g., oxadiazon) can be applied to an area (e.g., a field) that is under cultivation to selectively control weeds.

### Nucleic Acids and Polypeptides

As used herein, nucleic acids can include DNA and RNA, and includes nucleic acids that contain one or more nucleotide analogs or backbone modifications. A nucleic acid can be single stranded or double stranded, which usually depends upon its intended use and, in some instances, can encode a polypeptide. The sequences of two or more nucleic acids or two or more polypeptides can be described as having a percent sequence identity (e.g., a first sequence (e.g., a query) can have at least 50%, 55%, 60%, 65%, 70%, 75%, 80%, 85%, 90%, 91%, 92%, 93%, 94%, 95%, 96%, 97%, 98%, or 99% sequence identity to a second sequence (e.g., a subject)).

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

It would be appreciated by a skilled artisan that identifying and changing one or more amino acids requires that the context of a sequence, sometimes due to the context of a resulting structural feature, be preserved. For at least that reason, the numbering of the position referred to herein (i.e., position 212) is relative to the sequence of the Eleusine indica (L.) Gaertn (goosegrass) PPO1 protein, which is shown in SEQ ID NO:1. It would be understood, however, that any PPO1 or PPO2 protein, whether naturally occurring or modified or recombinant could be used as a unmodified (e.g., starting) sequence, i.e., reference sequence, although it would be understood that the numerical position may change from that referred to herein if a different reference sequence is used.

The context of a sequence, or the position of one or more amino acids in one sequence relative to another, typically is determined using a sequence alignment algorithm (e.g., Altschul et al., 1997, Nucleic Acids Res., 25:3389 3402 as incorporated into BLAST (basic local alignment search tool) programs, available at ncbi.nlm.nih.gov on the World Wide Web). BLAST or similar algorithms can be used to align two sequences (e.g., to identify the residue at a “corresponding” position, even if the two sequences differ, for example, in length), to identify motifs or consensus sequences, and/or to determine percent sequence identity between two or more sequences (nucleic acid or amino acid). As used herein, “default parameters” used when comparing two sequences are the default parameters using the BLAST algorithm (Version BLAST+ 2.10.1) as implemented at blast.ncbi.nlm.nih.gov on the World Wide Web on Sep. 23, 2020. For aligning protein sequences, the default parameters are BLASTP: parameters automatically adjusted for short input sequences; expect threshold: 10; word size: 3; max matches in a query range: 0; matrix: BLOSUM62; gap costs: existence 11, extension 1; compositional adjustments: conditional compositional score matrix adjustment; and no filters or masks). For aligning nucleic acid sequences, the default parameters are BLASTN: parameters automatically adjusted for short input sequences; expect threshold: 10; word size: 28; max matches in a query range: 0; match/mismatch scores: 1, −2; gap costs: linear; filter: low complexity regions; and mask: for lookup table only.

Changes can be introduced into a nucleic acid molecule, thereby leading to changes in the amino acid sequence of the encoded polypeptide. For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

As used herein, a “purified” polypeptide is a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the polypeptides and naturally occurring molecules with which it is naturally associated. Since a polypeptide that is chemically synthesized is, by nature, separated from the components that naturally accompany it, a synthetic polypeptide is “purified.”

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

A vector containing a nucleic acid (e.g., a nucleic acid that encodes a polypeptide) also is provided. Vectors, including expression vectors, are commercially available or can be produced by recombinant DNA techniques routine in the art. A vector containing a nucleic acid can have expression elements operably linked to such a nucleic acid, and further can include sequences such as those encoding a selectable marker (e.g., an antibiotic resistance gene). A vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6×His tag, glutathione S-transferase (GST))

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins. As used herein, operably linked means that a promoter or other expression element(s) are positioned in a vector relative to a nucleic acid in such a way as to direct or regulate expression of the nucleic acid.

Vectors as described herein can be introduced into a host cell. As used herein, “host cell” refers to the particular cell into which the nucleic acid is introduced and also includes the progeny of such a cell that carry the vector. A host cell can be any prokaryotic or eukaryotic cell. For example, nucleic acids can be expressed in bacterial cells such as E. coli, or in insect cells, yeast cells, plant cells or mammalian cells (such as Chinese hamster ovary cells (CHO) or COS cells). Suitable host cells are known to those skilled in the art. Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, N.Y.; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid.

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, N.Y.; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed. Simply by way of example, high stringency conditions typically include a wash of the membranes in 0.2×SSC at 65° C.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, Calif.).

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

### Plants and Methods of Making

Hybrids, varieties, lines, or cultivars are provided that have a mutation in one or more endogenous nucleic acids described herein (e.g., PPO1 or PPO2). As described herein, plants having a mutation in one or more of the endogenous nucleic acids (e.g., PPO1 or PPO2) can exhibit herbicide resistance, specifically oxadiazole resistance, compared to a corresponding plant lacking the mutation under corresponding growing conditions).

Methods of making a plant having a mutation are known in the art. Mutations can be random mutations or targeted mutations. For random mutagenesis, plant cells can be mutagenized using, for example, a chemical mutagen, ionizing radiation, or fast neutron bombardment (see, e.g., Li et al., 2001, Plant J., 27:235-42). Representative chemical mutagens include, without limitation, nitrous acid, sodium azide, acridine orange, ethidium bromide, and ethyl methane sulfonate (EMS), while representative ionizing radiation includes, without limitation, x-rays, gamma rays, fast neutron irradiation, and UV irradiation. The dosage of the mutagenic chemical or radiation is determined experimentally for each type of plant tissue such that a mutation frequency is obtained that is below a threshold level characterized by lethality or reproductive sterility. The number of M₁ generation seed or the size of M₁ plant populations resulting from the mutagenic treatments are estimated based on the expected frequency of mutations.

For targeted mutagenesis, representative technologies include TALEN technology (see, for example, Li et al., 2011, Nucleic Acids Res., 39(14):6315-25), zinc-finger technology (see, for example, Wright et al., 2005, The Plant J, 44:693-705), and CRISPR technology (see, for example, Mali et al., 2013, Nature Methods, 10:957-63). To accomplish CRISPR technology, nucleic acids encoding an endonuclease (e.g., Cas9 endonuclease, a Cpf1 endonuclease), a guide RNA and a nucleic acid modification template that includes the desired nucleic acid modification in the PPO nucleic acid sequence (i.e., to result in an A212T substitution in the encoded PPO polypeptide) can be incorporated into a vector and administered to a subject as described herein. Similarly, to accomplish TALEN technology, a nucleic acid encoding a TALEN (e.g., dimeric transcription factor/nuclease) can be incorporated into a vector and administered to a subject as described herein. Likewise, to accomplish zinc-finger nuclease technology, a nucleic acid encoding a custom DNA endonuclease (e.g., a heterodimer in which each subunit contains a zinc finger domain and a FokI endonuclease domain) can be incorporated into a vector and administered to a subject as described herein. Each of these technologies are available commercially; see, for example, Caribou Biosciences or CRISPR Therapeutics or Editas Medicine; Cellectis Bioresearch or Life Technologies; and Sangamo BioSciences or Sigma Aldrich Chemical Co., respectively. Under the appropriate circumstances and in the presence of the proper nucleic acids and polypeptides, gene editing can occur such that the A212T mutation described herein is introduced into a PPO sequence. See, for example, U.S. Pat. Nos. 8,697,359; 8,889,418; 8,999,641; US 2014/0068797; Li et al. (2011, Nucleic Acids Res., 39(14):6315-25); and Wright et al. (2005, The Plant J., 44:693-705).

A mutation in a nucleic acid disclosed herein (i.e., A212T) results in herbicide resistance, specifically oxadiazole resistance, in a plant carrying the mutation. Suitable types of mutations include, without limitation, insertions of nucleotides, deletions of nucleotides, or transitions or transversions. In some instances, a mutation is a point mutation; in some instances, a mutation encompasses multiple nucleotides. In some cases, a sequence includes more than one mutation or more than one type of mutation.

Polypeptides can include particular sequences that determine where the polypeptide is located within the cell, within the membrane, or outside of the cell. Target peptide sequences often are cleaved (e.g., by specific proteases that recognize a specific nucleotide motif) after the polypeptide is localized to the appropriate position. For example, PPO1 sequences typically include a chloroplast transit peptide, whereas PPO2 sequences typically include a mitochondrial transit peptide.

Following mutagenesis, M₀ plants are regenerated from the mutagenized cells and those plants, or a subsequent generation of that population (e.g., M₁, M₂, M₃, etc.), can be screened for a mutation in a sequence of interest (e.g., PPO1 or PPO2). Screening for plants carrying a mutation in a sequence of interest can be performed using methods routine in the art (e.g., hybridization, amplification, combinations thereof) or by evaluating the phenotype of the plants (e.g., for oxadiazole resistance). Generally, the presence of a mutation in one or more of the nucleic acid sequences disclosed herein (e.g., PPO1 or PPO2) results in oxadiazole resistance compared to a corresponding plant (e.g., having the same varietal background) lacking the mutation under corresponding growing conditions.

Herbicide resistant plants refer to plants in which an application of an amount of herbicide on the plant at concentrations and rates which are typically employed by the agricultural community to kill weeds in the field does not significantly affect or kill the plant, wherein a wild-type plant of the same species would be significantly affected and/or killed by the corresponding application of the herbicide. A plant may be naturally resistant to a particular herbicide, or a plant may be rendered herbicide resistant as a result of genetic engineering, such as for example, selective breeding; gene editing; and/or the introduction of a transgene within the genome of the plant. As used herein, a “herbicide resistant plant” refers to a plant containing a mutant PPO sequence as described herein that confers herbicide tolerance when provided to a heterologous plant. It would be understood that a plant that is herbicide resistant may show some minimal impact from the application of the herbicide (e.g., a moderate alteration in the growth and/or development, signs or symptoms associated with stress or disease), but one of skill in the art can readily distinguish between plants that are resistant to a herbicide and plants that are susceptible to a herbicide.

In addition, as used herein, statistical significance refers to a p-value of less than 0.05, e.g., a p-value of less than 0.025 or a p-value of less than 0.01, using an appropriate measure of statistical significance, e.g., a one-tailed two sample t-test.

An M₁ plant may be heterozygous for a mutant allele and exhibit a wild type phenotype. In such cases, at least a portion of the first generation of self-pollinated progeny of such a plant exhibits a wild type phenotype. Alternatively, an M₁ plant may have a mutant allele and exhibit a mutant phenotype. Such plants may be heterozygous and exhibit a mutant phenotype due to a phenomenon such as dominant negative suppression, despite the presence of the wild type allele, or such plants may be homozygous due to independently induced mutations in both alleles.

A plant carrying a mutant allele can be used in a plant breeding program to create novel and useful cultivars, lines, varieties and hybrids. Thus, in some embodiments, an M₁, M₂, M₃ or later generation plant containing at least one mutation is crossed with a second plant, and progeny of the cross are identified in which the mutation(s) is present. It will be appreciated that the second plant can contain the same mutation as the plant to which it is crossed, a different mutation, or be wild type at the locus. Additionally or alternatively, a second plant can exhibit a desired phenotypic trait such as, for example, disease resistance; high yield; high grade index; curability; curing quality; mechanical harvestability; holding ability; leaf quality; height; maturation; stalk size; and leaf number per plant.

Breeding is carried out using known procedures. DNA fingerprinting, SNP or similar technologies may be used in a marker-assisted selection (MAS) breeding program to transfer or breed mutant alleles into other lines, varieties or cultivars, as described herein. Progeny of the cross can be screened for a mutation using methods described herein, and plants having a mutation in a nucleic acid sequence disclosed herein (e.g., PPO1 or PPO2) can be selected. For example, plants in the F₂ or backcross generations can be screened using a marker developed from a sequence described herein or a fragment thereof, using one of the techniques listed herein. Plants also can be screened for herbicide resistance, specifically for oxadiazole resistance, and those plants having one or more of such phenotypes, compared to a corresponding plant that lacks the mutation, can be selected. Plants identified as possessing the mutant allele and/or the mutant phenotype can be backcrossed or self-pollinated to create a second population to be screened. Backcrossing or other breeding procedures can be repeated until the desired phenotype of the recurrent parent is recovered.

Successful crosses yield F₁ plants that are fertile and that can be backcrossed with one of the parents if desired. In some embodiments, a plant population in the F₂ generation is screened for the mutation using standard methods (e.g., PCR with primers based upon the nucleic acid sequences disclosed herein). Selected plants are then crossed with one of the parents and the first backcross (BC₁) generation plants are self-pollinated to produce a BC₁F₂ population that is again screened for the mutation or the herbicide-resistant phenotype. The process of backcrossing, self-pollination, and screening is repeated, for example, at least four times until the final screening produces a plant that is fertile and reasonably similar to the recurrent parent. This plant, if desired, is self-pollinated and the progeny are subsequently screened again to confirm that the plant contains the mutation and exhibits herbicide resistance, specifically oxadiazole resistance. Breeder's seed of the selected plant can be produced using standard methods including, for example, field testing, genetic analysis, and/or confirmation of the phenotype.

The result of a plant breeding program using the mutant plants described herein are novel and useful cultivars, varieties, lines, and hybrids. As used herein, the term “variety” refers to a population of plants that share constant characteristics which separate them from other plants of the same species. A variety is often, although not always, sold commercially. While possessing one or more distinctive traits, a variety is further characterized by a very small overall variation between individual with that variety. A “pure line” variety may be created by several generations of self-pollination and selection, or vegetative propagation from a single parent using tissue or cell culture techniques. A “line,” as distinguished from a variety, most often denotes a group of plants used non-commercially, for example, in plant research. A line typically displays little overall variation between individuals for one or more traits of interest, although there may be some variation between individuals for other traits.

Depending on the plant, hybrids can be produced by preventing self-pollination of female parent plants (i.e., seed parents) of a first variety, permitting pollen from male parent plants of a second variety to fertilize the female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by cytoplasmic male sterility (CMS), nuclear male sterility, genetic male sterility, molecular male sterility wherein a transgene inhibits microsporogenesis and/or pollen formation, or self-incompatibility. Female parent plants containing CMS are particularly useful. In embodiments in which the female parent plants are CMS, the male parent plants typically contain a fertility restorer gene to ensure that the F₁ hybrids are fertile. In other embodiments in which the female parents are CMS, male parents can be used that do not contain a fertility restorer. F₁ hybrids produced from such parents are male sterile. Male sterile hybrid seed can be interplanted with male fertile seed to provide pollen for seed-set on the resulting male sterile plants.

Varieties, lines and cultivars described herein can be used to form single-cross F₁ hybrids. In such embodiments, the plants of the parent varieties can be grown as substantially homogeneous adjoining populations to facilitate natural cross-pollination from the male parent plants to the female parent plants. The F₂ seed formed on the female parent plants is selectively harvested by conventional means. One also can grow the two parent plant varieties in bulk and harvest a blend of F₁ hybrid seed formed on the female parent and seed formed upon the male parent as the result of self-pollination. Alternatively, three-way crosses can be carried out wherein a single-cross F₁ hybrid is used as a female parent and is crossed with a different male parent. As another alternative, double-cross hybrids can be created wherein the F₁ progeny of two different single-crosses are themselves crossed. Self-incompatibility can be used to particular advantage to prevent self-pollination of female parents when forming a double-cross hybrid.

A mutant sequence as described herein can be overexpressed in plants, if so desired. Therefore, transgenic plants are provided that are transformed with a nucleic acid molecule described herein (e.g., PPO1 or PPO2) or a portion thereof under control of a promoter that is able to drive expression in plants (e.g., a plant promoter). As discussed herein, a PPO1 or PPO2 nucleic acid used in a plant expression vector can have a different sequence than the PPO1 or PPO2 sequence described herein, which can be expressed as a percent sequence identity or based on the conditions under which sequences hybridize. As an alternative to using a full-length sequence, a portion of the sequence can be used that encodes a polypeptide fragment having the desired functionality, or lack thereof.

Methods of introducing a nucleic acid (e.g., a heterologous nucleic acid) into plant cells are known in the art and include, for example, particle bombardment, Agrobacterium-mediated transformation, microinjection, polyethylene glycol-mediated transformation (e.g., of protoplasts, see, for example, Yoo et al. (2007, Nature Protocols, 2(7):1565-72)), liposome-mediated DNA uptake, or electroporation. Following transformation, the transgenic plant cells can be regenerated into transgenic plants. As described herein, expression of the transgene results in plants that exhibit herbicide resistance, specifically oxadiazole resistance, relative to a plant not expressing the transgene. The regenerated transgenic plants can be screened for exhibit herbicide resistance, specifically oxadiazole resistance, compared to a corresponding non-transgenic plant, and can be selected for use in, for example, a breeding program as discussed herein.

Following transformation, the transgenic cells can be regenerated into transgenic plants, which can be screened for exhibit herbicide resistance, specifically oxadiazole resistance, and plants having such herbicide resistance, compared to a corresponding non-transgenic plant, can be selected and used, for example, in a breeding program as discussed herein.

Using the methods described herein, an oxadiazole-resistant tomato cell or seed; an oxadiazole-resistant tobacco cell or seed; an oxadiazole-resistant oil seed rape cell or seed; an oxadiazole-resistant flax cell or seed; an oxadiazole-resistant soybean cell or seed; an oxadiazole-resistant sunflower cell or seed; an oxadiazole-resistant sugar beet cell or seed; an oxadiazole-resistant alfalfa cell or seed; and an oxadiazole-resistant cotton cell or seed are provided.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1-E. indica Biotypes and Growth Condition

Three E. indica biotypes from different locations were used in this study. The R1 biotype was collected in Country Club of Virginia, Richmond, Va., and the R2 biotype was from River Bend Golf, New Bern, N.C. These two biotypes were previous confirmed resistant to preemergence application of oxadiazon, but have not been screened to other PPO inhibitors. The S biotype was collected from the Alabama Agricultural Experiment Station, Plant Breeding Unit, Tallassee, Ala., which was confirmed susceptible to PPO inhibitors. Seeds of the mature plants were harvested, air dried and stored in the 4° C. freezer until planted in the greenhouse.

Greenhouse conditions were 30±3° C. at day/night temperature and ˜70% average relative humidity. The E. indica seeds were placed on the soil surface and lightly covered with sand in 28 cm*20 cm flats and watered as needed daily to ensure germination. Two weeks after emergence, seedlings were separated and transplanted to in individual 10-cm pots (volumes=0.5 L). Pots were filled with surface horizon of Marvyn sandy loam (fine-loamy, kaolinitic, thermic Typic Kanhapludults) soil with pH 6.4 and 1.2% organic matter. The plants were irrigated three times daily for 2 min with overhead irrigation and fertilized once at one week after transplanting at approximately 50 kg N ha⁻¹ with Scott's Miracle-Gro All-Purpose fertilizer (The Scotts Miracle-Gro Co. Marysville, Ohio). Plants were 5-10 cm in height with 3-5 tillers in size at the time of herbicide treatment.

### Example 2-Herbicide Rate Screening Assay

Herbicide Treatment

Herbicide treatments were foliar-applied at 280 L/ha using an enclosed spray chamber with a single 8002E nozzle (TeeJet Spray Systems Co, Wheaton, Ill.) at 32 PSI. Four herbicides were selected for the experiment: oxadiazon (Ronstar FLO, Bayer Environmental Sci., Research Triangle Park, N.C.), sulfentrazone (Dismiss, FMC Corporation, Philadelphia, Pa.), flumioxazin (Sureguard, Valent Corp., Walnut Creek, Calif.) and lactofen (Cobra, Valent Corp., Walnut Creek, Calif.). All the herbicides are from different herbicide chemical families of PPO inhibitors: oxadiazoles, triazolinone, N-phenylphthalimide and diphenyl ether, respectively. Herbicides were applied at 7 different rates based on each herbicide label rate: oxadiazon ranging from 0.14 to 8.96 kg/ha, sulfentrazone from 0.07 to 4.50 kg/ha, flumioxazin from 0.08 to 5.70 kg/ha, and lactofen from 0.029 to 1.75 kg/ha. A non-treated control (0 kg/ha) was included. 192 plants of each biotype were tested and experiments were conducted as completely random design, three replications for two runs.

Data Analysis

The visual injury rating scores per plant at 14 days after treatment (DAT) were recorded, where the visual injury rating scores were based on a 0 to 100 scale, which 0 is equated to no phytotoxicity and 100 is equated to complete control. Data subjected to ANOVA analysis at a significance level of P<0.05 using the PROC GLM procedure of SAS 9.4 (SAS Institute Inc., Cary, N.C.). All the herbicide rates were log transformed to make equal spacing between the herbicide treatments in order to facilitate regression analysis. The non-treated control was transformed to equal spacing based on the log rates of each herbicide, respectively. Data were fitted to a sigmoidal model using SigmaPlot 10.2 (Systat Software Inc., London, UK) using a sigmoidal function (Equation 1).

y=a/(1+e\hat{ }(−((x−x0)/b))) (1)

In this fit sigmoidal model, where y represents E. indica visual damage relative to non-treated control (%), x represents the log-transformed herbicide rates (kg/ha), three parameters (a, b, x0) represents the y intercept. This sigmoidal equation was used to calculate the inhibition rate at 50% (I₅₀) and 90% (I₉₀) relative to the non-treated control of each herbicide for each biotype, and the 95% confidence intervals (α=0.05) were calculated for regression parameters.

### Example 3—Transcriptome Analysis and PPO Gene Identification

RNA Extraction

Total RNA of three E. indica biotypes (R1, R2 and S) were extracted from fresh leaves using the RNeasy plant kit (QIAGEN, Aarhus, Denmark). Leaves of each biotypes were taken from three well-growth plants. The quality and quantity of the total RNA was assessed by NanoDrop 2000 Spectrophotometer (Thermo Fisher Scientific Co., Waltham, Mass.) and determined with gel electrophoresis before RNA_Seq analysis. cDNA was synthesized from high-quality total RNA using the ProtoScript first strand cDNA synthesis kit (New England Biolabs Inc. Ipswich, Mass.).

Transcriptome Assembly and Protein Alignment

RNA-Seq libraries from R1 and R2 E. indica biotypes were generated at the Genomic Service Laboratory at the Hudson Alpha Institute for Biotechnology (Cummings Research Park, Huntsville, Ala.). The raw sequencing reads of R1 and R2 E. indica biotypes have been submitted in the National Center for Biotechnology Information (NCBI) Sequence Read Archive (SRA) database as Accession Nos. SAMN10817169, SAMN10817194, respectively. The RNA-Seq dataset of the S biotype was acquired from the NCBI-SRA database under Accession No. SRR 1560465. Similarly, a previously published draft genome assembly of the S biotype was downloaded from NCBI as Accession No. SAMN09001275. Raw RNA-Seq reads R1, R2 and S were assembled using the following pipeline. Adaptor contamination and unqualified reads were removed via Trimmomatic-0.32, then the trimmed reads were quality checked with FastQC and de novo assembly with Trinity 2014-04-13pl. Three assembly datasets were annotated with the NCBI nonredundant (Nr) protein database (blast.ncbi.nlm.nih.gov on the World Wide Web) with NCBI-BLAST-2.2+. The Nr blast results were processed to identify and compare to reference PPO1 and PPO2 downloaded from the NCBI database (Table 4). Read extractions and mapping to identify single nucleotide polymorphisms and other related mutations and all the contig reads of the blast PPO genes were extracted using bowtie2 (bowtie-bio.sourceforge.net/bowtie2/ on the World Wide Web) and samtools (samtools.sourceforge.net/ on the World Wide Web), and compared with the draft genome annotation scaffold in the CLC Genomics Workbench 6.5.2 (QIAGEN, Aarhus, Denmark). The protein alignment of PPO1 and PPO2 were using clustalX2 and ENDscript 3.0 server.

TABLE 4
List of PPO1 and PPO2 in other species and accession
numbers downloaded from NCBI database

| | | Gene | NCBI |
|---|---|---|---|
| Abbreviations | Scientific Name | Name | Accession No |
| At_PPO1 | Arabidopsis thaliana | PPO1 | NP_192078 |
| Nt_PPO1 | Nicotiana tabacum | PPO1 | BAA34713 |
| Si_PPO1 | Setaria italica | PPO1 | XP_004967639 |
| Sb_PPO1 | Sorghum bicolor | PPO1 | XP_002455484 |
| AmT_PPO1 | Amaranthus tuberculatus | PPO1 | ABD52324 |
| At_PPO2 | Arabidopsis thaliana | PPO2 | NP_001190307 |
| AmT_r_PPO2 | Amaranthus tuberculatus | PPO2 | ABD52328 |
| | (resistant) | | |
| AmT_s_PPO2 | Amaranthus tuberculatus | PPO2 | ABD52326 |
| | (susceptible) | | |
| Si_PPO2 | Setaria italica | PPO2 | XP_004976030 |
| Sb_PPO2 | Sorghum bicolor | PPO2 | XP_002446710 |
| Ap_PPO2 | Amaranthus palmeri | PPO2 | ATE88443 |
| St_PPO2 | Solanum tuberosum | PPO2 | XP_006356026 |
| Gm_PPO2 | Glycine max | PPO2 | NP_001236376 |
| Nt_PPO2 | Nicotiana tabacum | PPO2 | NP_001312887 |

cDNA Sequencing

Two pairs of oligonucleotide primers were designed based on the sequences of PPO1 and PPO2, respectively. The primers for PPO1 are 5′-ATG GTC GCC ACG CCC GCA AT-3′ (chlF) (SEQ ID NO:6) and 5′-CTT GTA GGC GTA CTT GGT CAA G-3′ (chlR) (SEQ ID NO:7) and 1587 bp PCR product. The primers for PPO2 are 5′-ATG GCG GGC TCC GAC GAC AC-3′ (mitF) (SEQ ID NO:8) and 5′-ATG TGA ACT GTC ATG CTT TGT GC-3′ (mitR) (SEQ ID NO:9), and 1533 bp PCR product. The PCR reaction system contained up to 1 μg cDNA, 200 nM of the forward and reverse primers, 200 μM dNTPs and 1.0 U of Taq polymerase (New England Biolabs Inc., Ipswich, Mass.) with a 1× concentration of standard Taq buffer in a final volume of 25 μL. After initial denaturation of the cDNA at 95° C. for 1 min; there were 35 cycles of 30 s at 95° C., 1 min at 58° C. and 2 min at 68° C.; then a final extension at 68° C. for 10 min. PCR products were extracted by gel electrophoresis, sequenced, and analysis conducted using the CLC Genomics Workbench 6.5.2 (QIAGEN, Aarhus, Denmark).

### Example 4—E. coli Functional Assay

Two putative PPO-inhibitor resistant (R-) and susceptible (S-) plasmids were created to test the role of the chloroplast-targeted PPO1 in the E. indica biotypes. The PPO1 from R1, R2 and S E. indica biotypes were cloned into the pBAD-TOPO expression vector using the pBAD TOPO™ TA Expression kit (Invitrogen, Carlsbad, Calif.), respectively. The PPO1 product was amplified using the same PCR primers and PCR reaction system as cDNA sequencing, so that the PPO1 translation began at the ATG start codon. Three different pBAD-TOPO PPO1 constructs were created and sequenced to confirm they were identical with the PPO1 gene from R1, R2 and S E. indica biotypes and there were no other nucleotide polymorphisms in the cloning experiment. R- and S-PPO1 plasmids were used to transform a hemG mutant Escherichia coli strain SASX38 by electroporation. The SASX38 mutant strain was grown on LB medium supplemented with 10 μg/mL hematin. Expression of the PPO1 in the transformed colonies of the SASX38 mutant strain were induced on LB medium with 2% L-arabinose. Growth and survival of the transformed colonies of E. coli with PPO1 from R1, R2 and S E. indica biotypes (marked as: R1, R2 and S, respectively) and a non-transformed control strain (NT), were tested on three different media: LB alone, LB medium supplied with 10 μg/mL hematin, or with the PPO inhibitor oxadiazon from 10 μM to 200 μM, and incubated at 37° C. for 20 h.

### Example 5—Recombinant Expression, Purification, and In Vitro Inhibition Studies of PPO1

Effects of A212T substitution were studied using the E. indica backbone. The wild-type E. indica PPO1 and the E. indica PPO1 A212T variants were synthesized de novo and subcloned into pRSetB plasmid (Invitrogen, Carlsbad, Calif.). The complete description of expression and purification of E. indica PPO1 and E. indica PPO1 A212T variant proteins were referenced to the method described for PPO2 by Rangani et al. (2019, Frontiers in Plant Sci., 10:568). Six PPO inhibitors, belonging to five different chemical families, were evaluated on the PPO enzyme activity at a concentrations ranging from 5.00×10⁻⁵M to 5.12×10⁻¹²M. Oxadiazon, sulfentrazone, saflufenacil and lactofen are from the class of oxadiazole, triazolinone, pyrimidinedione, and diphenyl ether, respectively. Flumioxazin and trifludimoxazin are from the same chemical family, N-phenylphthalimide. The concentration of the wild-type PPO1 activity and variant PPO1 A212T activity 50% (IC50 values) reduced by the inhibitors was estimated using non-linear regression procedures, based on each inhibitor. The assay was replicated twice.

### Example 6—Computational Modeling

A homology model of wild-type E. indica PPO1 (S-PPO1 model) was built using the workflow of Schrödinger's Prime (Schrödinger Release 2019-1: Prime, Schrödinger, LLC, New York, N.Y.). Default settings and protein preparation settings were applied. As a reference structure, an in-house protein crystal structure of Amaranthus tuberculatus PPO2 was selected. The sequence similarity between E. indica PPO1 and A. tuberculatus PPO2 is 29.2% in total, and 46.4% within the binding site (all residues within 5 A to the modeled ligand). Oxadiazon was modeled into the binding site using binding mode information of known in-house co-crystal structures and docking functionality of the modeling program Molecular Operating Environment (MOE, 2019.01: Chemical Computing Group, Montreal, QC, Canada). The predicted poses were refined by local minimization of the ligand and the receptor structure. To examine the effect of the A212T mutation on oxadiazon binding, the homology model of E. indica PPO1 was modified into a second model (R-PPO1 model), where Ala212 was virtually mutated to Thr212.

### Example 7—Herbicide Rate Screening

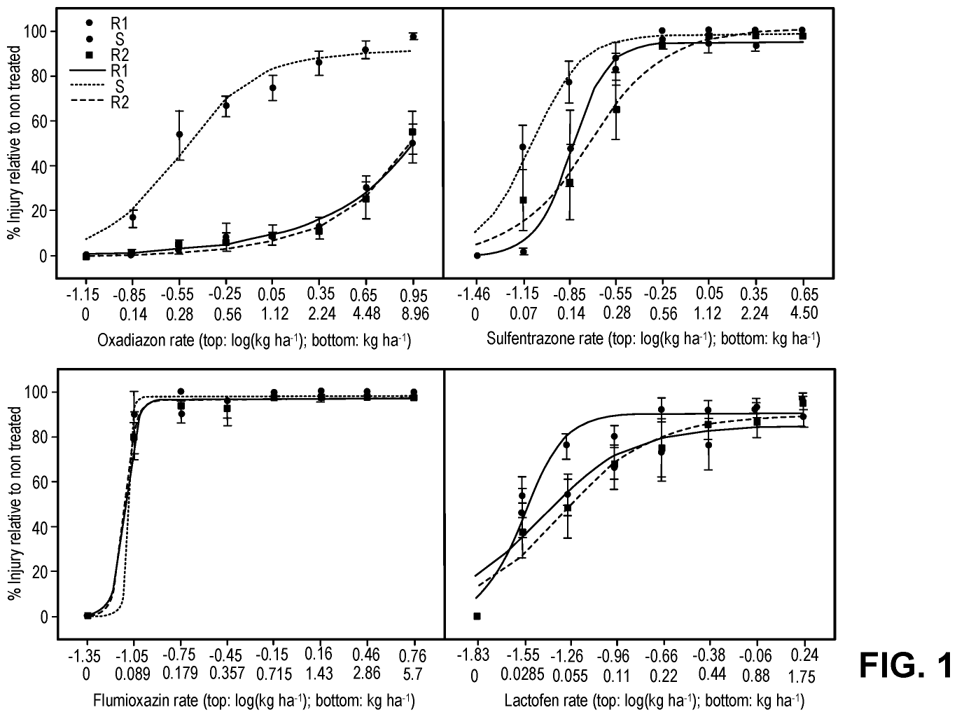

Herbicide rate responses focus on comparison of oxadiazon, lactofen, flumioxazin and sulfentrazone on the S and R biotypes. The resistant and susceptible biotypes are not obviously different before herbicide screening. A dose response curve was developed to model the individual biotype response to each tested herbicide (FIG. 1). Labelled rate applications of flumioxazin (0.357 kg/ha), sulfentrazone (0.28 kg/ha), and lactofen (0.22 kg/ha) controlled the resistant biotypes (R1 and R2) approximately 70 to 100%, while the labelled rate application of oxadiazon (2.24 kg/ha) provided less than 10% control. Little to no difference was observed between the R1, R2 and S E. indica biotype response to flumioxazin, lactofen and sulfentrazone at these labelled rates. However, a significant difference was observed between the resistant (R1 and R2), and susceptible (S) response to oxadiazon. Oxadiazon at 2.24 kg/ha controlled the S biotype >80% compared to <20% for the R1 and R2 biotypes. Difference in the R1 and R2 compared to the S response to oxadiazon was consistent for all oxadiazon concentrations tested, while little difference was observed between R1 and R2 compared to S at lower rates of lactofen and sulfentrazone, 0.055 kg/ha and 0.14 kg/ha, respectively, in dose response curves. Such a response at lower rates may indicate a slight resistance to lower rates in the oxadiazon-resistant R1 and R2 E. indica biotypes. No such differences between R1, R2, and S were observed with respect to the flumioxazin rates.

I₅₀ and I₉₀ values of the different PPO-inhibitors for each E. indica biotype were calculated based on the model for the curve and the best fit equation (Table 5 and Table 6). The 150 values of the S biotype for oxadiazon was 0.32 kg/ha, while I₅₀ value of the R1 biotype and R2 biotype for oxadiazon was 8.15 kg/ha and 8.88 kg/ha, respectively. The I₉₀ values of the R1 biotype and the R2 biotype for oxadiazon was 14.60 kg/ha and 18.29 kg/ha, respectively, while the I₉₀ value of the S biotype for oxadiazon was 1.56 kg/ha. This indicates that the previously confirmed pre-emergence oxadiazon resistant E. indica biotypes still displayed up to 20-fold increased resistance than the susceptible biotype when post-emergence was applied with oxadiazon. No significant differences in response to flumioxazin, sulfentrazone and lactofen were observed for Iso and I₉₀ values between the R1, R2 and S biotypes (Table 6). These two previous confirmed oxadiazon resistant E. indica biotypes had no significant cross-resistance to other PPO inhibitors except to oxadiazon.

TABLE 5
Predictive model with sigmoidal equation for percent injury in response to increasing rates of four different protoporphyrinogen
oxidase (PPO) inhibitors relative to a non-treated control within three Eleusine indica biotypes. Parameter estimate
and parameter estimate 95% confidence interval (CI) are presented as means of model comparison.

| | | Equation^{b} | | | | | | | |
|---|---|---|---|---|---|---|---|---|---|
| | | y = a/(1 + | | Parameter estimates and confidence interval | | | | | |
| Herbicides | Biotypes^{a} | exp(−(x − x₀)/b)) | R² | a | 95% CI | b | 95% CI | x₀ | 95% CI |
| Oxadiazon | S | y = 91.25/(1 + | 0.97 | 91.25 | (81.65, 100.65) | 0.25 | (0.13, 0.37) | −0.54 | (−0.86, −0.41) |
| | | exp(−(x − (−0.54))/0.25)) | | | | | | | |
| | R1 | y = 9925.40/(1 + | 0.99 | 9925.40 | (9747.04, 10103.76) | 0.53 | (0.26, 0.80) | 3.75 | (−93.36, 100.87) |
| | | exp(−(x − 3.75)/0.53)) | | | | | | | |
| | R2 | y = 64371.40/(1 + | 0.99 | 64371.40 | (64167.56, 64575.24) | 0.43 | (0.15,0.70) | 3.99 | (−590.17, 598.15) |
| | | exp(−(x − 3.99)/0.43)) | | | | | | | |
| Sulfentrazone | S | y = 98.21/(1 + | 0.98 | 98.21 | (91.88, 104.55) | 0.16 | (0.09, 0.23) | −1.10 | (−1.18, −1.02) |
| | | exp(−(x − (−1.10))/0.16)) | | | | | | | |
| | R1 | y = 95.12/(1 + | 0.99 | 95.12 | (91.13, 99.10) | 0.12 | (0.08, 0.16) | −0.84 | (−1.03, −0.65) |
| | | exp(−(x − (−0.84))/0.12)) | | | | | | | |
| | R2 | y = 100.90/(1 + | 0.98 | 100.90 | (92.45, 109.34) | 0.25 | (0.16, 0.33) | −0.73 | (−0.83, −0.63) |
| | | exp(−(x − (−0.73))/0.25)) | | | | | | | |
| Flumioxazin | S | y = 98.00/(1 + | 0.99 | 98.00 | (95.41, 100.59) | 0.02 | (−0.11, 0.14) | −1.09 | (−1.28, −0.90) |
| | | exp(−(x − (−1.09))/0.02)) | | | | | | | |
| | R1 | y = 96.53/(1 + | 0.99 | 96.53 | (93.75, 99.31) | 0.04 | (−0.08, 0.17) | −1.11 | (−1.30, −0.92) |
| | | exp(−(x − (−1.11))/0.04)) | | | | | | | |
| | R2 | y = 96.81/(1 + | 0.99 | 96.81 | (94.23, 99.39) | 0.04 | (−0.16, 0.23) | −1.11 | (−1.41, −0.81) |
| | | exp(−(x − (−1.11))/0.04)) | | | | | | | |
| Lactofen | S | y = 90.29/(1 + | 0.97 | 90.29 | (84.29, 96.29) | 0.13 | (0.06, 0.20) | −1.52 | (−1.60, −1.45) |
| | | exp(−(x − (−1.52))/0.13)) | | | | | | | |
| | R1 | y = 84.65/(1 + | 0.86 | 84.65 | (68.30, 101.00) | 0.29 | (0.02, 0.57) | −1.44 | (−1.71, −1.17) |
| | | exp(−(x − (−1.44))/0.29)) | | | | | | | |
| | R2 | y = 89.20/(1 + | 0.95 | 89.20 | (77.82, 100.58) | 0.30 | (0.14, 0.46) | −1.31 | (−1.48, −1.14) |
| | | exp(−(x − (−1.31))/0.30)) | | | | | | | |

TABLE 6
Estimated rate of different PPO inhibiting herbicides required to reduce
Eleusine indica biotype by 50% (I50) and 90% (I90) based on the injury
scores collected 14 days after treatment. 95% confidence intervals (CI)
at I50 and I90 values are provided as means of comparison

| | | Visual Control/Injury ^{b} | | | |
|---|---|---|---|---|---|
| Herbicides | Biotype^{a} | I₅₀ (kg ha⁻¹) | 95% CI | I₉₀ (kg ha⁻¹) | 95% CI |
| Oxadiazon | S | 0.32 | (0.05, 1.18) | 1.56 | (1.13, 3.24) |
| | R1 | 8.15 | (7.18, 9.12) | 14.60 | (13.63, 15.57) |
| | R2 | 8.88 | (8.13, 9.64) | 18.29 | (17.53, 19.05) |
| Sulfentrazone | S | 0.08 | (0.00, 0.34) | 0.19 | (0.00, 0.45) |
| | R1 | 0.15 | (0.00, 0.44) | 0.32 | (0.03, 0.61) |
| | R2 | 0.18 | (0.00, 0.51) | 0.63 | (0.31, 0.95) |
| Flumioxazin | S | 0.08 | (0.04, 0.12) | 0.09 | (0.04, 0.13) |
| | R1 | 0.08 | (0.04, 0.12) | 0.10 | (0.06, 0.14) |
| | R2 | 0.08 | (0.04, 0.13) | 0.10 | (0.05, 0.15) |
| Lactofen | S | 0.03 | (0.00, 0.12) | 0.17 | (0.08, 0.26) |
| | R1 | 0.04 | (0.00, 0.18) | 0.51 | (0.36, 0.66) |
| | R2 | 0.06 | (0.00, 0.21) | 0.50 | (0.35, 0.65) |

### Example 8—PPO Gene Isolation and Target Site Assessment

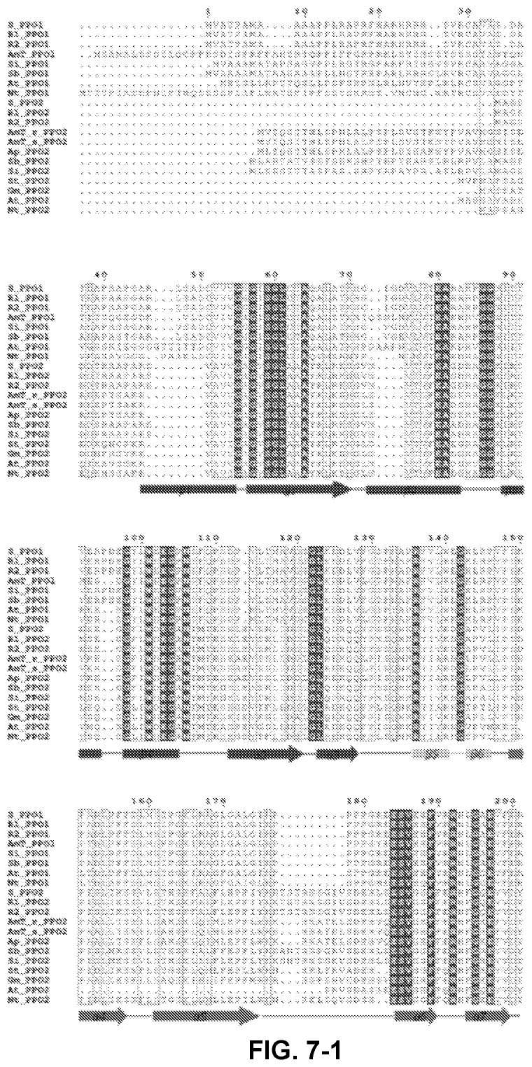

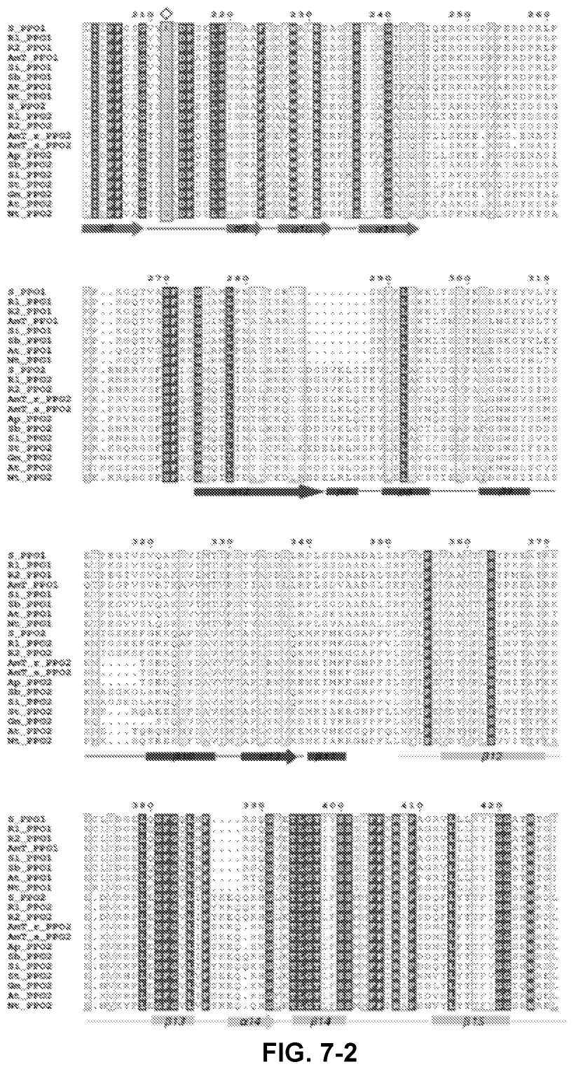

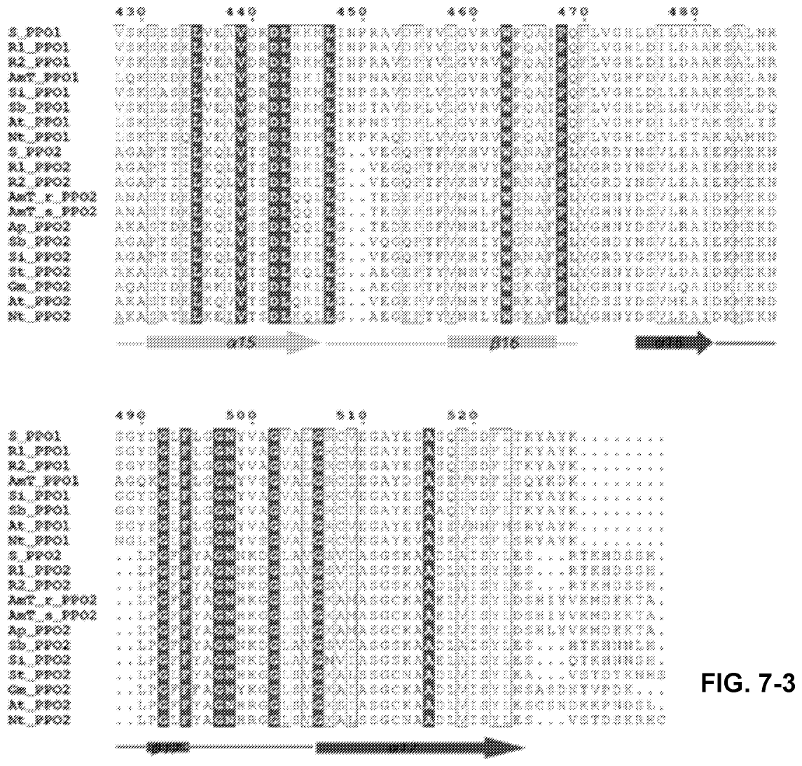

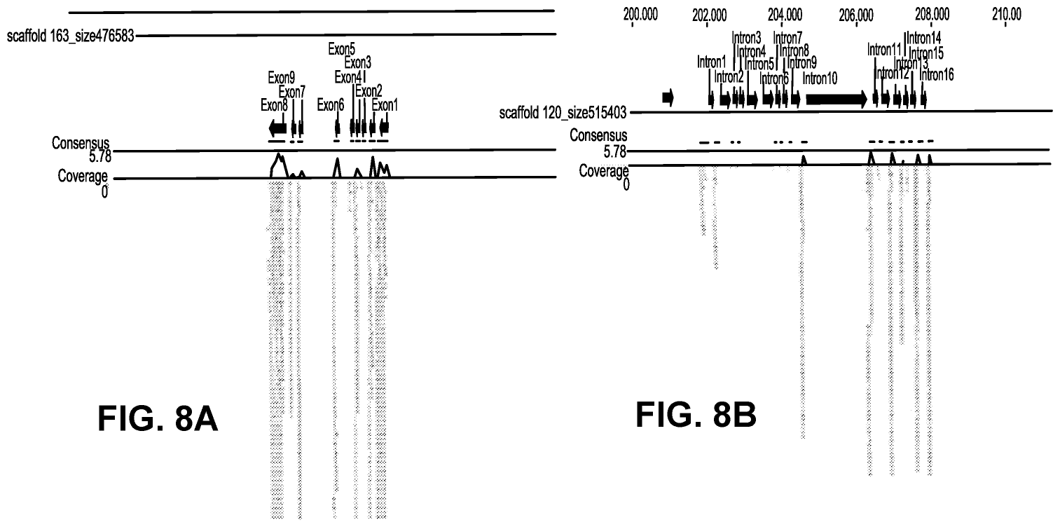

Two related genes in E. indica, PPO1 and PPO2, were isolated based on the transcriptome analysis and cDNA sequencing. In the S biotype, the related gene reads of PPO1 and PPO2 were extracted and mapped with the E. indica assembly draft genome (Table 7, FIG. 8). The genomic DNA (gDNA) of chloroplast-targeted PPO1 of E. indica has 3816 bp, containing 9 exons and 8 introns (Table 8); the mitochondrial-targeted PPO2 gDNA has 6043 bp, including 17 exons and 16 introns (Table 9). Comparison of the translated sequences of the PPO1 and PPO2 of the S E. indica biotype reveal 23.35% amino acid identity and 38.50% similarity (FIG. 7). These results showed that there are significant sequence differences between PPO1 and PPO2 in E. indica. The gDNA and cDNA of PPO1 and PPO2 in S biotype have been submitted to NCBI GenBank database as Accession Nos. MK573537, MK040459, MK573539, MK573538, respectively.

TABLE 7
The reads and gene assembly of PPO1 and PPO2 of the RNA datasets of Eleusine indica biotypes.

| | | | | PPO1^{b} | | | | | PPO2^{b} | | | | |
|---|---|---|---|---|---|---|---|---|---|---|---|---|---|
| | | Trimmed | | | Extrac- | | | Amino | | Extrac- | | | Amino |
| Biotype^{a} | Reads | Read | Trimming | Reads | tions | Exons | Length | Acid | Reads | tion | Exons | Length | Acid |
| S | 136513042 | 113353352 | 83.03% | — | — | — | — | — | — | — | — | — | — |
| R1 | 38990558 | 33333028 | 85.49% | 111352 | 0.33% | 9 | 3816 bp | 529 | 8864 | 0.02% | 17 | 6043 bp | 511 |
| R2 | 59063288 | 51089744 | 86.50% | 105520 | 0.21% | — | — | — | 7216 | 0.01% | — | — | — |

TABLE 8
The structure of the Eleusine indica chloroplast-targeted PPO1 gene for
the exons and introns based on the mapping results analysis.

| | | Size | % | | | Size | % | |
|---|---|---|---|---|---|---|---|---|
| Exon^{a} | Location | (bp) | (G + C) | Intron | Location | (bp) | (G + C) | 5′ donor & 3′ acceptor seq |
| exonl | 1-354 | 354 | 74.85 | intron1 | 355-435 | 81 | 54.32 | CC/GTACGC . . . GCAG/GT |
| exon2 | 436-621 | 186 | 67.20 | intron2 | 622-783 | 162 | 44.09 | CA/GTTCGT . . . TCAG/GG |
| exon3 | 784-868 | 85 | 62.35 | intron3 | 869-963 | 95 | 33.68 | AG/GTGCTT . . . TAAG/GT |
| exon4 | 964-1111 | 148 | 47.30 | intron4 | 1112-1183 | 72 | 31.94 | CC/GTAAGA . . . CCAG/CC |
| exon5 | 1184-1264 | 81 | 54.32 | intron5 | 1265-1690 | 426 | 34.74 | AG/GTTTAT . . . ACAG/GT |
| exon6 | 1691-1862 | 172 | 43.02 | intron6 | 1863-2930 | 1068 | 35.54 | CA/GTAAGT . . . ACAG/AG |
| exon7 | 2931-3090 | 160 | 45.00 | intron7 | 3091-3236 | 146 | 26.71 | AG/GTAAAT . . . AAAG/GA |
| exon8 | 3237-3340 | 104 | 45.19 | intron8 | 3341-3516 | 176 | 33.52 | AG/GTTCTA . . . GCAG/AG |
| exon9 | 3517-3816 | 300 | 48.67 | | | | | |

^{a}The exons begin at the start codon, and end at the stop codon.

TABLE 9
The structure of the Eleusine indica mitochondria-targeted PPO2 gene for
the exons and introns based on the mapping results analysis.

| | | Size | % | | | Size | % | |
|---|---|---|---|---|---|---|---|---|
| Exon^{a} | Location | (bp) | (G + C) | Intron | Location | (bp) | (G + C) | 5' donor & 3' acceptor seq |
| exon 1 | 1-68 | 68 | 79.41 | intron 1 | 69-217 | 149 | 58.39 | AG/GTGAGT . . . GCAG/TG |
| exon 2 | 218-353 | 136 | 64.71 | intron 2 | 354-681 | 328 | 42.99 | TG/GTGAGC . . . GCAG/AC |
| exon 3 | 682-747 | 66 | 40.91 | intron 3 | 748-850 | 103 | 34.95 | AT/GTATGT . . . TCAG/CC |
| exon 4 | 851-901 | 51 | 47.06 | intron 4 | 902-1005 | 104 | 36.54 | TG/GTAATA . . . TCAG/AT |
| exon 5 | 1006-1062 | 57 | 31.58 | intron 5 | 1063-1395 | 333 | 31.53 | AG/GTATGT . . . GCAG/TT |
| exon 6 | 1396-1481 | 86 | 36.05 | intron 6 | 1482-1828 | 347 | 35.16 | AG/GTGAGT . . . GCAG/TG |
| exon 7 | 1829-1865 | 37 | 48.65 | intron 7 | 1866-1983 | 118 | 29.66 | AG/GTGAGT . . . ACAG/GT |
| exon 8 | 1984-2049 | 66 | 42.42 | intron 8 | 2050-2218 | 169 | 37.87 | CT/GTGAGT . . . GCAG/AT |
| exon 9 | 2219-2259 | 41 | 36.59 | intron 9 | 2260-2534 | 275 | 28.36 | AA/GTAAGT . . . CCAG/GT |
| exon 10 | 2535-2667 | 133 | 42.86 | intron 10 | 2668-4322 | 1655 | 40.06 | AG/GTAACC . . . TCAG/TC |
| exon 11 | 4323-4511 | 189 | 42.33 | intron 11 | 4512-4605 | 94 | 37.23 | CA/GTAAGG . . . TCAG/GC |
| exon 12 | 4606-4680 | 75 | 41.33 | intron 12 | 4681-4907 | 227 | 33.04 | AG/GTCAGG . . . GCAG/GT |
| exon 13 | 4908-5034 | 127 | 45.67 | intron 13 | 5035-5204 | 170 | 36.47 | TG/GTAGGT . . . ATAG/GT |
| exon 14 | 5205-5313 | 109 | 45.87 | intron 14 | 5314-5392 | 79 | 29.11 | AC/GTATAT . . . GCAG/GA |
| exon 15 | 5393-5467 | 75 | 41.33 | intron 15 | 5458-5618 | 151 | 34.44 | AA/GTAAGT . . . TCAG/GC |
| exon 16 | 5619-5725 | 107 | 49.53 | intron 16 | 5726-5930 | 205 | 37.56 | AG/GCAAGC . . . ATAG/GA |
| exon 17 | 5931-6043 | 113 | 45.13 | | | | | |

^{a}The exons begin at the start codon, and end at the stop codon.

### Example 9—A Novel Single Amino Acid Substitution A212T in PPO1 as the Hypothesis of Oxadiazon Resistance in the E. indica Biotypes

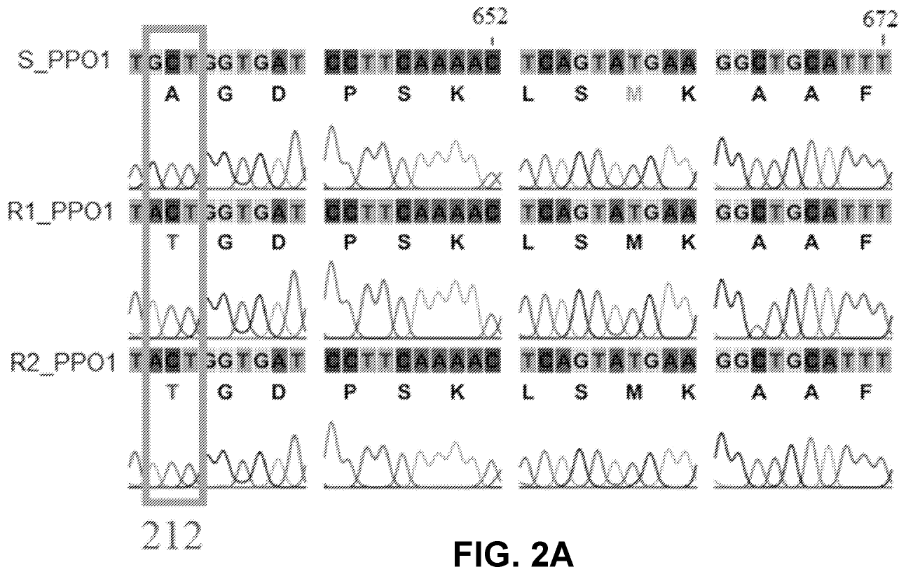

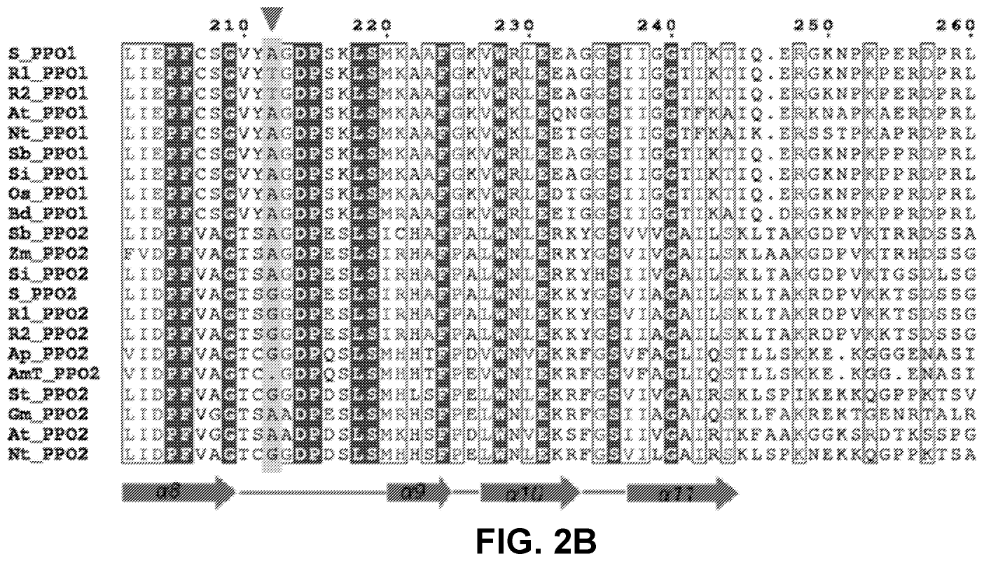

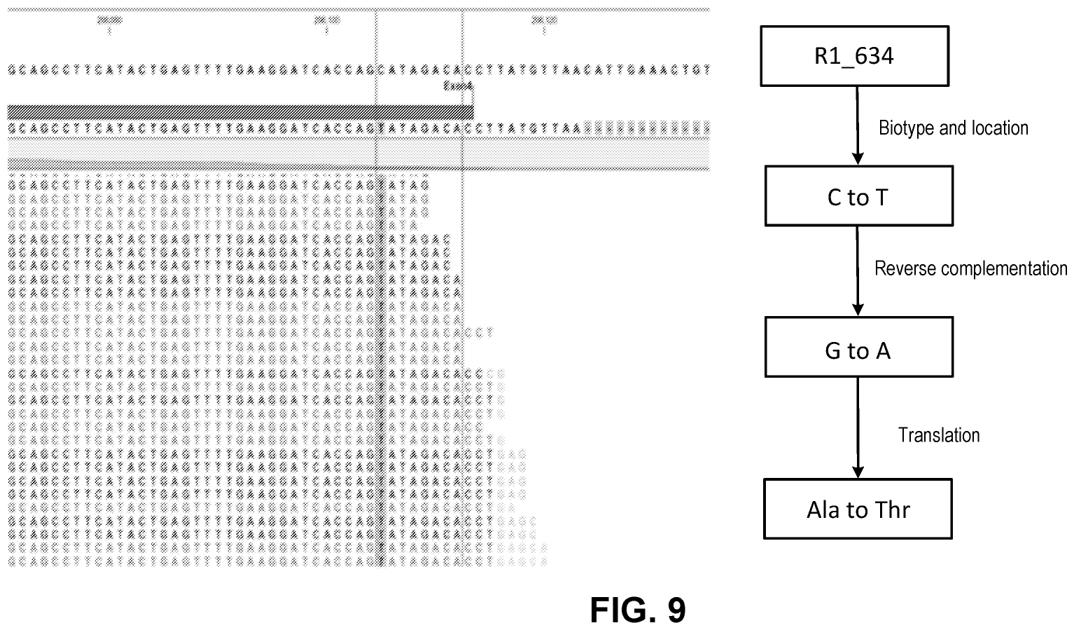

The sequence of the two unique genes of the three biotypes (S, R1 and R2) were aligned and mapped to the E. indica genomic DNA to identify any possible single nucleotide polymorphisms (SNPs) (FIG. 8 and FIG. 9). There were no nonsynonymous substitutions in the R1 biotype PPO2 gene, but two amino acid substitutions, V207I and T303A, were identified only in the R2 biotype (FIG. 7). Protein alignments of E. indica PPO2 with the A. tuberculatus PPO2 indicated that Val207 (V235 in A. tuberculatus) and Thr303 (Ser324 in A. tuberculatus) are not part of the catalytic domain (FIG. 7), suggesting that these two substitutions in R2 biotype mitochondrial PPO2 maybe not confer resistance. However, some SNP nonsynonymous substitutions were identified in the PPO1 when comparing S, R1 and R2 E. indica (GenBank Accession Nos. MK040459, MK040460, MK040461, respectively). There was a single amino acid substitution, A212T, identified in both R1 and R2 E. indica biotypes (Table 1), and two other amino acid substitutions only in R2 biotype, T283A and K366M (Table 1). Resequencing of this locus confirmed the presence of the SNP and the resulting amino acid substitution (FIG. 2A). Protein alignment of the amino acid sequences of PPO1 and PPO2 indicates that alanine 212 in PPO1 is highly conserved in other species (FIG. 2B), and it is synonymous with glycine 210 in PPO2, which was also highly conserved in other species and previously confirmed as the causal resistance mechanism to PPO inhibitors in A. tuberculatus when lacking G210. This indicated that the substitution A212T in E. indica PPO1 as the possible mutation conferring resistance to PPO inhibitor oxadiazon. The cDNA of PPO1 and PPO2 in R1 and R2 biotypes were submitted to NCBI GenBank database as accession numbers: MK040460, MK040461, MN256107, MN256106, respectively.

TABLE 1
Identification of nucleotide polymorphisms using read mapping to assembled chloroplast
PPO1 contigs of both resistant and susceptible Eleusine indica biotypes. Amino
acid substitution comparisons between S, R1 and R2 E. indica biotypes

| | Amino | S* | | R1* | | R2* | |
|---|---|---|---|---|---|---|---|
| Nucleotide | acid | | Amino | | Amino | | Amino |
| position | position | Nucleotide | acid | Nucleotide | acid | Nucleotide | acid |
| 634 | 212 | G | Ala | A | Thr | A | Thr |
| 847 | 283 | A | Thr | A | Thr | G | Ala |
| 1097 | 366 | A | Lys | A | Lys | T | Met |

*E. indica biotype abbreviations: S, known susceptible biotype; R1, oxadiazon resistant biotype from Country Club of Virginia, Richmond, VA; R2, oxadiazon resistant biotype from River Bend Golf Course, New Bern, NC.

### Example 10—The R-PPO1 Confers Resistance to Oxadiazon in hemG Mutant E. coli Complementation Assay

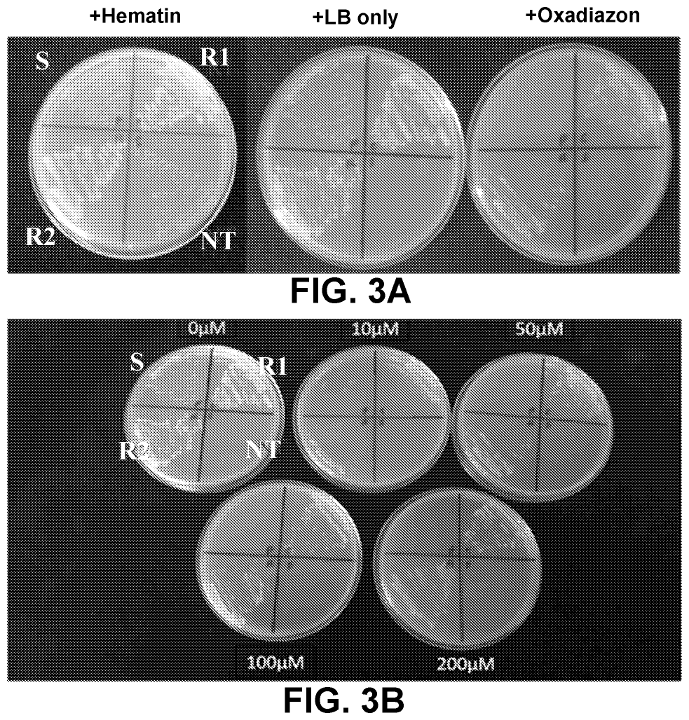

An E. coli functional assay using a mutant of the bacterial protoporphyrinogen IX oxidase-deficient, hemG, was implemented to compare the function of PPO1 from the R1, R2 and S biotypes in the presence of oxadiazon. The SASX38 mutant strain can grow when supplemented with exogenous heme (hematin) or an alternative source of PPO. The SASX38 E. coli strain was transformed with plasmids expressing the PPO1 genes of R1, R2 and S. All the transformed SASX38 strains were able to grow on LB medium without being supplemented with hematin, while the non-transformed SASX38 strain (NT) was unable to grow unless supplied with exogenous hematin (FIG. 3A), indicating that all the PPO1 genes encoded a functional protein. Five increasing oxadiazon concentrations (0, 10, 50, 100, and 200 μM) were evaluated, and the medium with 50 μM oxadiazon inhibited the growth of the E. coli transformed with the S PPO1 alleles, but the E. coli transformed with the PPO1 from the R1 and R2 E. indica alleles were able to grow at a concentration of up to 200 μM oxadiazon (FIG. 3B). This is sufficient evidence to conclude that the R1 and R2 PPO1 remains functional in the presence of oxadiazon and the A212T mutation is the most likely mechanism of resistance to oxadiazon in R1 and R2 E. indica biotypes since it is the only common amino acid change in PPO1, which suggest that the single amino acid change in PPO1 causes resistance to oxadiazon.

### Example 11—E. indica PPO1 A212T is Highly Resistant to Oxadiazon but not to Saflufenacil, Sulfentrazone, Lactofen, Flumioxazin and Trifludimoxazin

The above described greenhouse experiments, complementation assay and sequencing data suggest that the R1 and R2 alleles carry a mutation that endows resistance specifically to oxadiazon. Interestingly, both R1 and R2 alleles contain the A212T substitution. To test whether A212T in E. indica PPO1 is the main cause of the observed oxadiazon resistance, we used an in vitro activity assay to determine the inhibition potency (IC50) of oxadiazon towards recombinant wild-type E. indica PPO1 and the mutant A212T PPO1 enzymes. In addition, to test whether A212T leads to cross-resistance to other PPO-inhibiting herbicides in vitro, the assay was also performed with other PPO-inhibitors: saflufenacil, sulfentrazone, lactofen, flumioxazin and trifludimoxazin.

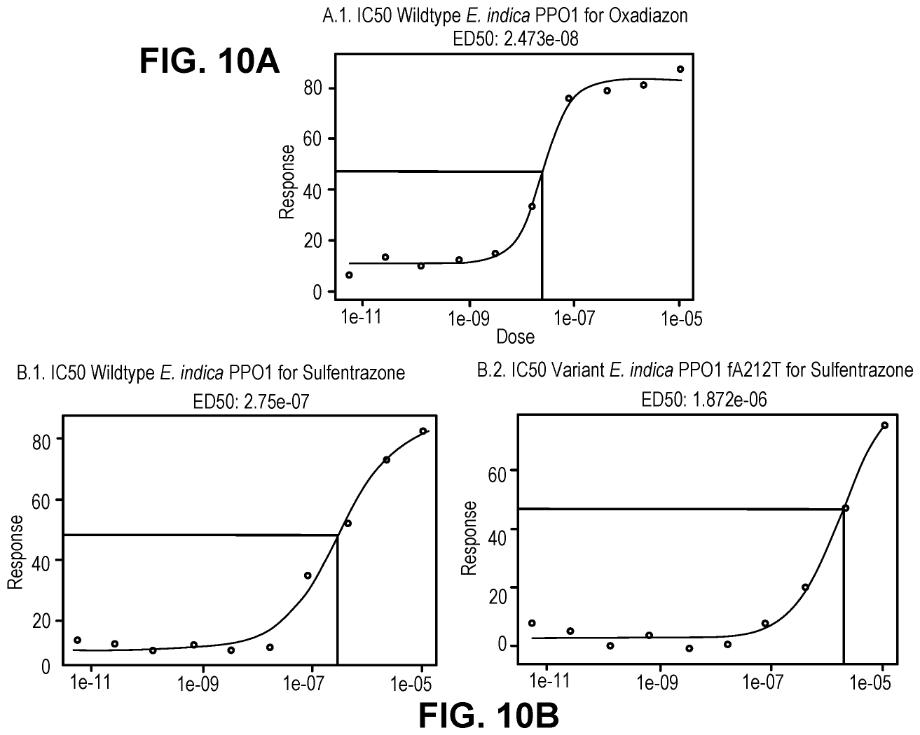

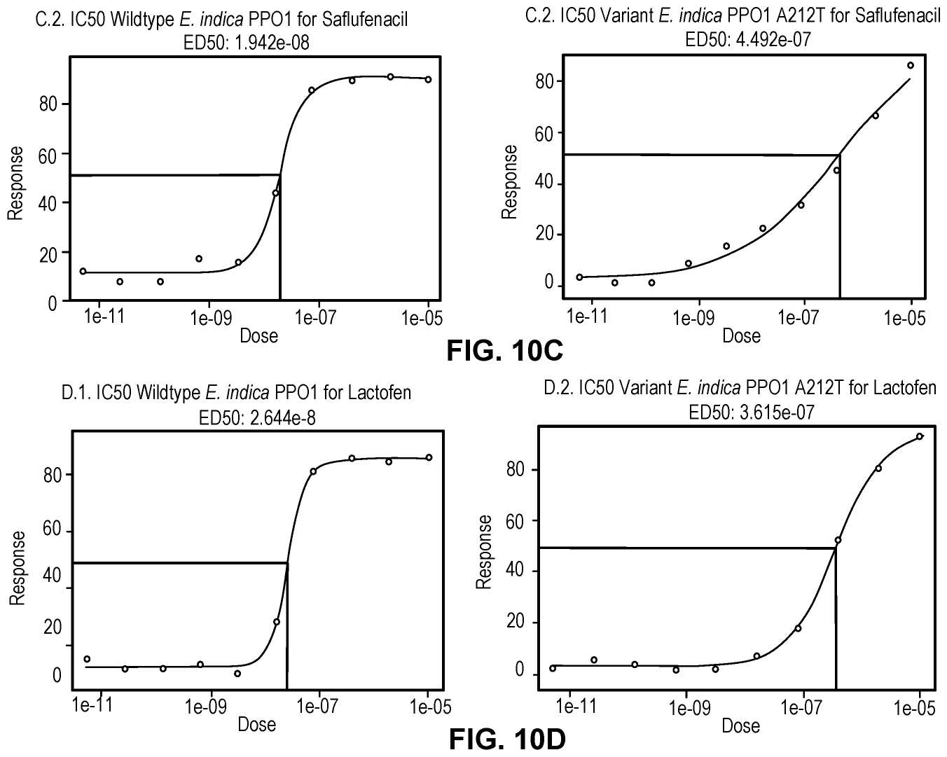

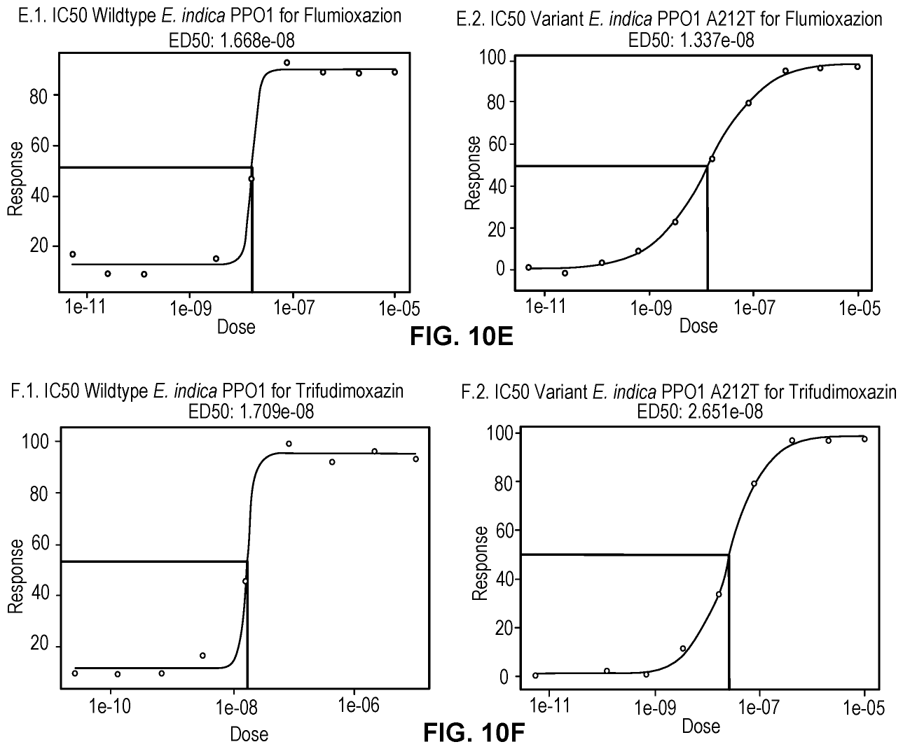

Oxadiazon strongly inhibited wild-type E. indica PPO1, exhibiting the classical dose response curve and an IC50 of 2.47×10⁻⁸ M (Table 2, FIG. 10). At the highest concentration of oxadiazon (1.00×10⁻⁵ M) more than 90% of the enzyme activity was inhibited (Table 2). In contrast, the A212T mutant E. indica PPO1 was so poorly inhibited, that no IC50 could be determined (>1.00×10⁻⁵ M) (Table 2). At the highest concentration of oxadiazon, only 16% of the A212T mutant E. indica PPO1 enzyme activity was inhibited. This result indicates that A212T confers high resistance to oxadiazon in vitro. Saflufenacil, lactofen, flumioxazin and trifludimoxazin strongly inhibited both the wild-type E. indica PPO1 and the A212T mutant E. indica PPO1 enzymes (Table 2). At the highest herbicide concentration, more than 90% of inhibition was achieved for both recombinant enzymes (Table 2). Sulfentrazone inhibition potency was slightly less towards the A212T mutant E. indica PPO1 when compared to the wild-type E. indica PPO1, exhibiting an IC50 of 1.87×10⁻⁶ M and 2.75×10⁻⁷ M, respectively. Among the tested PPO-inhibiting herbicides trifludimoxazin and flumioxazin were the most potent towards the A212T mutant E. indica PPO1 (Table 2).

TABLE 2
Effects of protoporphyrinogen oxidase (PPO) inhibitors on in vitro enzyme activity
of recombinant Eleusine indica wild-type PPO1 and variant A212T PPO1 enzyme.

| | | Wild-type | Variant | | Variant |
|---|---|---|---|---|---|
| | | E. indica | E. indica | Wild-type | E. indica |
| | | PPO1 | PPO1 A212T | E. indica | PPO1 |
| PPO chemical | | sensitivity | sensitivity | PPO1 % | A212T % |
| family | Herbicides | [IC50] (M) | [IC50] (M) | inhibition^{†} | inhibition^{†} |
| Oxadiazoles | Oxadiazon | 2.47 × 10⁻⁸ | >1.00 × 10⁻⁵ | >90% | 16% |
| Triazolinones | Sulfentrazone | 2.75 × 10⁻⁷ | 1.87 × 10⁻⁶ | >90% | 76% |
| Pyrimidinediones | Saflufenacil | 1.94 × 10⁻⁸ | 4.49 × 10⁻⁷ | >90% | >90% |
| Diphenyl ethers | Lactofen | 2.64 × 10⁻⁸ | 3.62 × 10⁻⁷ | >90% | >90% |
| N-Phenyl- | Flumioxazin | 1.67 × 10⁻⁸ | 1.34 × 10⁻⁸ | >90% | >90% |
| phthalimides | | | | | |
| | Trifludomoxazin | 1.71 × 10⁻⁸ | 2.65 × 10⁻⁸ | >90% | >90% |

^{†}% inhibition with the rate of the highest herbicide concentration 10⁻⁵ M.

### Example 12—Computational Modeling of the A212T Mutation in PPO1

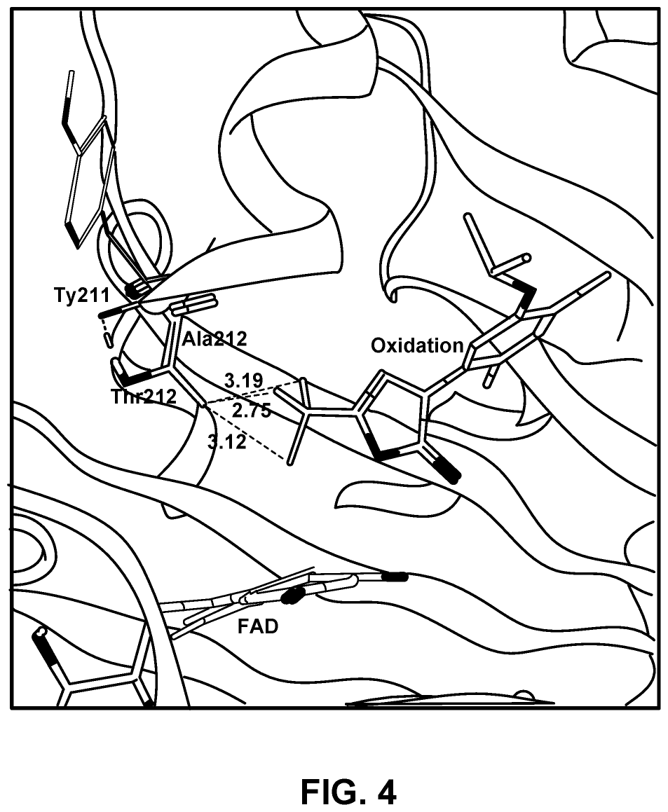

An in-house high resolution X-ray crystal structure of A. tuberculatus PPO2 was used as a template to model the consequence of the A212T mutation in E. indica PPOL. The modeled oxadiazon binding pose fits well into the binding site of the E. indica PPO1 wild-type homology model, forming many favorable Van der Waals interactions. In the homology model with the A212T mutation, this was not the case. The resulting visualization (FIG. 4) suggests the following hypothesis for A212T-induce resistance to oxadiazon. The threonine 212 can form a hydrogen bond with the carbonyl backbone of the neighboring tyrosine 211. This causes the C-gamma methyl group (—CH₃) to be very restrained in an orientation facing the oxadiazon binding. Due to clashes between the threonine C-gamma methyl (—CH₃) and tert-butyl group (—C4H9) of oxadiazon, thus the inhibitor, oxadiazon, is pushed out of the binding site (FIG. 4). Therefore, these repulsive interactions reduce the strength of enzyme-ligand binding energy, weakening the inhibition of PPO1 by oxadiazon.

### Example 13—Weed Control Screen

Research was conducted to evaluate the ability to control common weeds with oxadiazon (1.0 lb ai/a), glyphosate (1.0 lb ai/a), and glufosinate (1.0 lb ai/a) at common labeled rates used in agriculture. Herbicides were applied at a spray volume of 280 L/ha with no adjuvant added to any herbicide mixture. Weed control was rated on a 0 to 100 percent phytoxicity scale with 0 being no plant injury/phytotoxicity and 100 being complete plant dessication. Twenty-one weeds were evaluated for response to herbicides (Table 3). The weeds selected were a mixture of weeds common in agronomic and horticultural crops. Herbicide was applied to all species approximately 3 weeks after emergence. Plant phytotoxicity rated as percent control relative to the non-treated. Oxadiazon provided a similar level of weed control to glyphosate and glufosinate and even greater weed control on some species (Table 3). Oxadiazon controlled 13 of the 21 weeds evaluated at a level greater than or equal to 90%. Oxadiazon control 18 or 21 weeds greater than or equivalent to glyphosate—the most common herbicide used for agronomic weed control in the world.

TABLE 3
Percent control (0 to 100) of select weed species by the indicated herbicide

| Common Name | Scientific Name | Oxadiazon | Glufosinate | Glyphosate |
|---|---|---|---|---|
| silktree | Albizia julibrissin | 50 | 100 | 25 |
| sicklepod | Senna obtusifolia | 90 | 100 | 70 |
| showy crotalaria | Crotalaria spectabilis | 99 | 100 | 60 |
| hemp sesbania | Sesbania exaltata | 100 | 100 | 75 |
| cutleaf groundcherry | Physalis angulata | 70 | 100 | 99 |
| doveweed | Murdannia nudiflora | 0 | 100 | 80 |
| pitted morningglory | Ipomoea lacunosa | 100 | 100 | 60 |
| prickly sida | Sida spinosa | 90 | 100 | 60 |
| rice flatsedge | Cyperus iria | 60 | 60 | 90 |
| red morningglory | Ipomoea coccinea | 100 | 100 | 50 |
| cypressvine | Ipomoea quamoclit | 100 | 100 | 50 |
| morningglory | | | | |
| entireleaf morningglory | Ipomoea hederacea | 100 | 100 | 60 |
| smallflower | Jaquemontia tamnifolia | 90 | 100 | 40 |
| morningglory | | | | |
| glyphosate-resistant | Conyza candensis | 30 | 100 | 0 |
| horseweed | | | | |
| Carolina horsenettle | Solanum carolinense | 60 | 100 | 50 |
| cock's comb kyllinga | Kyllinga squamulata | 75 | 30 | 95 |
| johnsongrass | Sorghum halapense | 100 | 100 | 100 |
| goosegrass | Eleusine indica | 100 | 100 | 90 |
| velvetleaf | Abutilon theophrasti | 100 | 100 | 100 |
| smooth pigweed | Amaranthus hybridus | 100 | 100 | 90 |
| glyphosate resistant | Amaranthus palmeri | 75 | 100 | 35 |
| palmer amaranth | | | | |

### Example 14—Transgenic Tobacco and Soybean

The E. indica protoporphyrinogen oxidase (PPO1) open reading frame, with its endogenous transit peptide, was codon optimized (Genscript, Piscataway, N.J.) for soybean (Glycine max). The resultant codon optimized PPO sequence (SEQ ID NO: 5), along with the soybean ubiquitin promoter, coupled with its first intron (De La Torre et al., 2015, Plant Cell Rep., 34:111-20) and the transcriptional termination signal from the cauliflower mosaic virus 35S transcript were subsequently synthesized (Genscript). The synthesized PPO expression cassette was subcloned into the binary vector, pPZP212 (Hajdukiewicz et al., 1994, Plant Mol. Biol., 25:989-94), and the resultant binary vector was designated pPTN1513. A second plasmid was then assembled in which the synthesized PPO expression cassette was subcloned into the binary vector, pPTN1138, which harbors a bar gene (Thompson et al., 1987, EMBO, 6:2519-23) selectable marker regulated by the nopaline synthase promoter from A. tumefaciens. This final binary vector was designated pPTN1514.

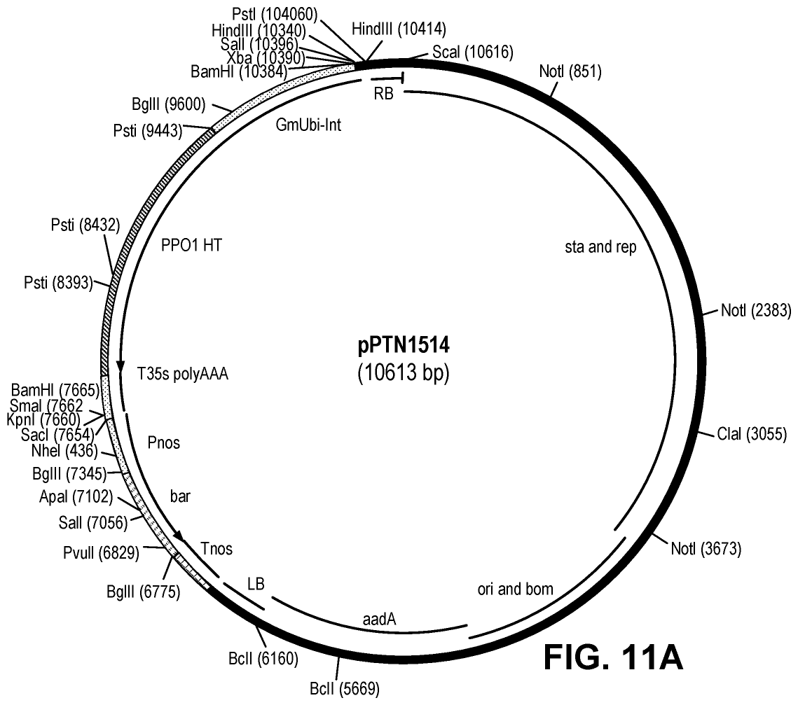

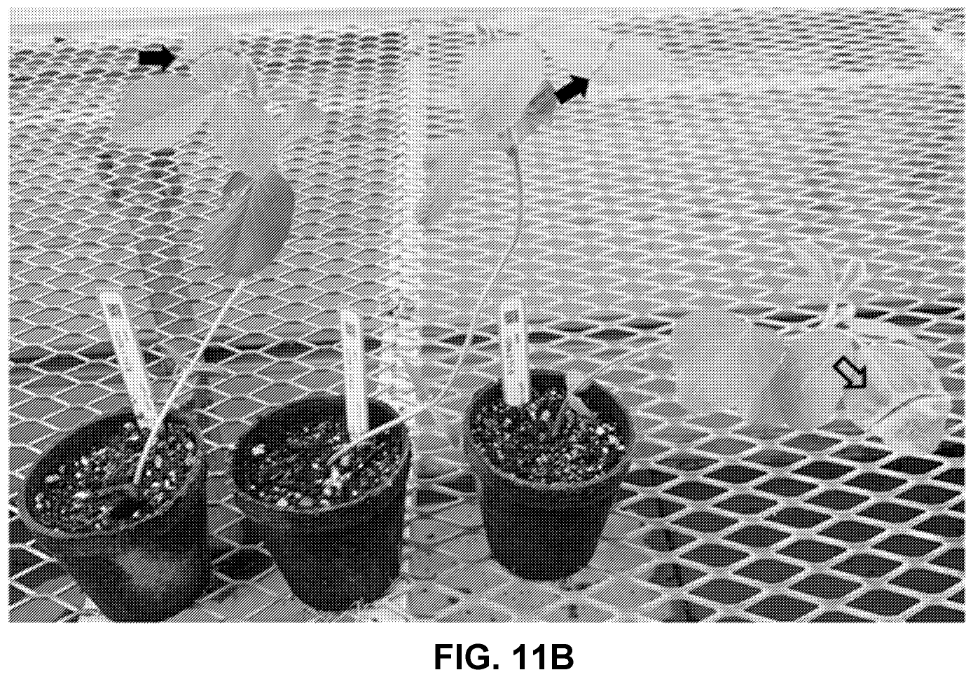

The binary vector pPTN1513 was mobilized into A. tumefaciens strain C58C1/pMP90 (Koncz et al., 1986, Mol. Gen. Genet., 204:383-96) and the resultant transconjugant used for tobacco (cv Xanthi) transformation following the protocol of Horsch et al. (Horsch et al., 1985, Science, 227:1229-31). The binary vector, pPTN1514 (FIG. 11A), was mobilized in A. tumefaciens strain EHA101 (Hood et al., 1986, J. Bacteriol., 168:1291-1301) and the resultant transconjugant used for soybean transformation (Zhang et al., 1999, Plant Cel Tiss. Org. Cult., 56:37-46).

Derived T1 soybean plants were first screened for tolerance to the selectable marker gene bar to monitor for presence or absence of the T-DNA element (FIG. 11B), and the plants carrying the T-DNA element along with null segregants and wild type soybean plants are subsequently phenotyped for oxadiazon tolerance.

Germination Tolerance Assay

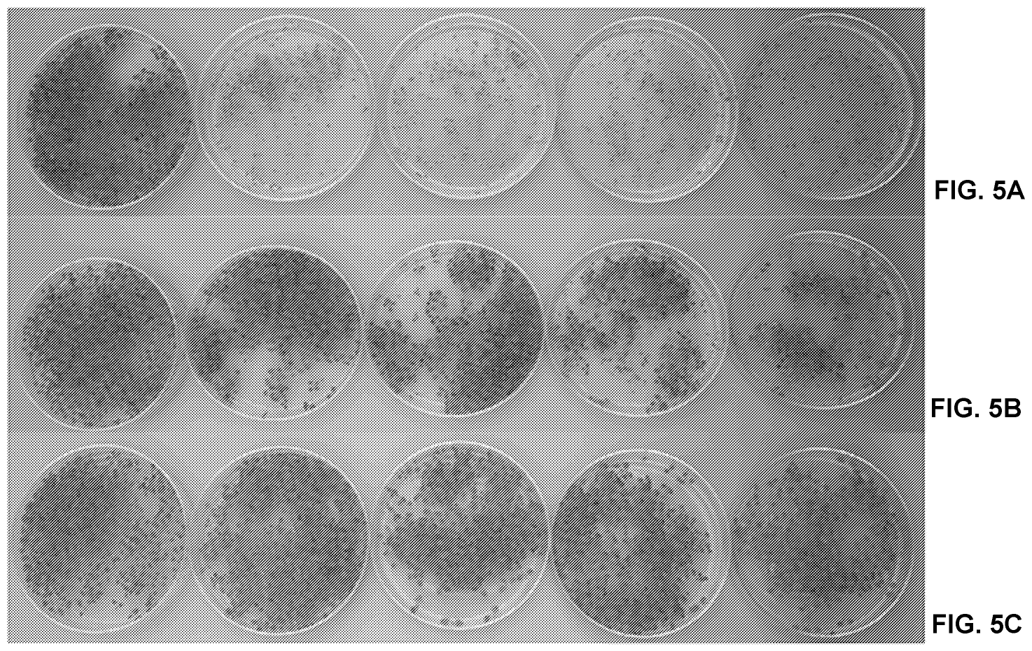

Seed collected from primary tobacco events, along with wild type non-transformed control seeds, were surfaced sterilized and plated onto MS medium supplemented with 0, 0.5 mg/l, 1.0 mg/l, 3 mg/l or 5 mg/l oxadiazon (Sigma). The plates were allowed to incubate at 24° C. under an 18-hour light regime for 13 days (FIG. 5).

Greenhouse/Field Plots

Plants were grown from a number of transformation events, and research was conducted to evaluate five of the lines containing the A212T amino acid substitution in PPO1 (T22, T26, T32, T33, and T38) for response to oxadiazon compared to a non-transformed line. Treatments included oxadiazon at 0.25, 0.50, 1.0, 2.0, and 4.0 lb ai/a, lactofen at 0.19 lb ai/a, glyphosate at 0.5 lb ai/a, and a non-treated check. Applications were made using a C02 pressurized backpack sprayer at a spray volume of 280 L/ha. No surfactant was added to the spray mixture.

Tobacco lines were germinated in potting soil and transplanted to individual pots and allowed to acclimate for two weeks prior to treatment. Soybean lines are similarly germinated. Treatments were applied approximately four weeks after seeding or three weeks after germination.

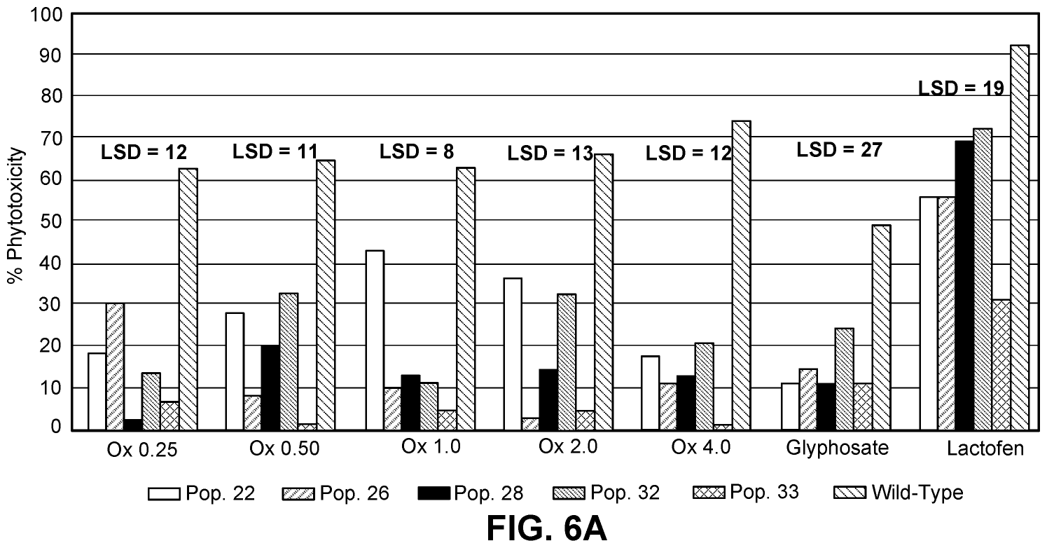

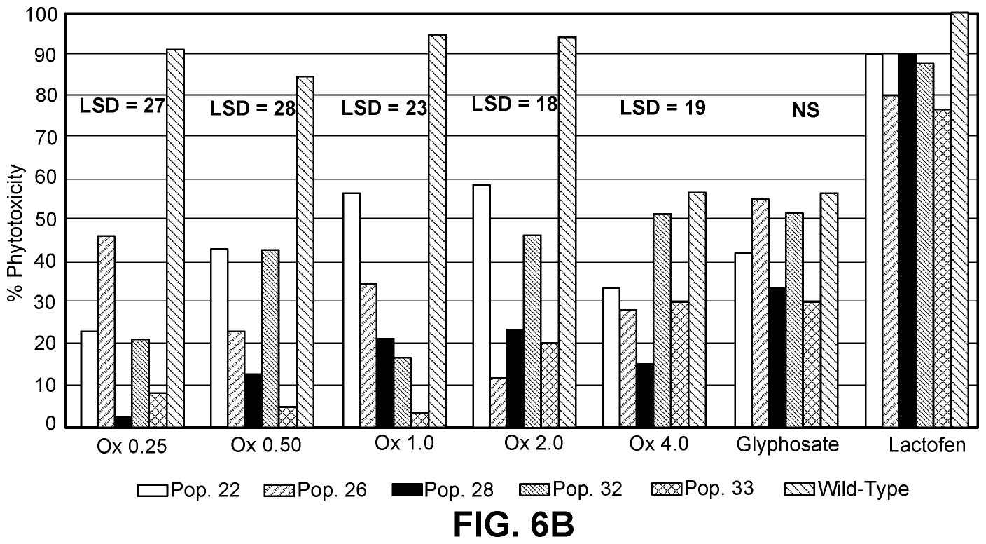

Plants were rated visually on a 0 to 100% scale where 0 is no visible plant injury or phytotoxicity and 100 is complete plant death or necrosis. By comparison, 50% injury is desiccation of half of the plant tissue relative to the non-treated. Treatments were rated at 3 and 7 days after treatment. See FIG. 6.

Oxadiazon is a fast-acting, non-selective herbicide that induced phytotoxic symptoms in 1 to 3 days, allowing for evaluation of plant response relatively soon after treatment.

### Example 15—Reference Sequences

PPO1 from Eleusine indica-chloroplast(QHA79696.1; GI: 1788579941)
(SEQ ID NO: 1)
MVATPAMAAA APPLRAPRFH ARHRRRSVRC AVASDATEAP AAPGARLSAD CVVVGGGISG
LCTAQALATK HGIGDVLVTE ARARPGGNIT TVERPDEGYL WEEGPNSFQP SDPVLTMAVD
SGLKEDLVFG DPNAPRFVLW EGKLRPVPSK PADLPFFDLM SIPGKLRAGL GALGIRPPPP
GREESVEEFV RRNLGAEVFE RLIEPFCSGV YAGDPSKLSM KAAFGKVWRL EEAGGSIIGG
TIKTIQERGK NPKPERDPRL PKPKGQTVAS FRKGLAMLPN AITSRLGSKV KLSWKLTSIT
KSDSKGYVLV YETPEGIVSV QAKSVIMTIP SYVASDILRP LSSDAADALS RFYYPPVAAV
TVSYPKEAIR KECLIDGELQ GFGQLHPRSQ GVETLGTIYS SSLFPNRAPA GRVLLLNYIG
GATNIGIVSK SESELVEAVD RDLRKMLINP RAVDPYVLGV RVWPQAIPQF LVGHLDILDA
AKSALNRSGY DGLFLGGNYV AGVALGRCVE GAYESASQIS DFLTKYAYK
PPO1 from Eleusine indica-chloroplast (MK573537.1)
(SEQ ID NO: 2)
atggtcgcca cgcccgcaat ggccgccgcc gcgccgccgc tccgagcgcc gcgattccat
gcgcgtcacc gccgcagaag cgtgcgctgc gcggtggcca gcgacgccac cgaggcgccg
gccgcgcccg gcgcgcggct gtccgcggac tgcgtcgtgg tgggcggcgg catcagcggc
ctctgcacgg cgcaggcgct ggccacgaaa cacggcatcg gagacgtact tgtcacggag
gcccgcgccc gccccggcgg caacatcacc accgtcgagc gccccgacga ggggtactta
tgggaggaag ggcccaacag cttccagccc tccgaccccg tcctcaccat ggccgtacgc
ttctcgctcc cttcttctat tcctcttaca gtacatttct cgtgaacgct gaatgaactg
ggcgcgcgcg cgcaggtgga cagcggactc aaggaggact tggtgtttgg agacccgaac
gcgccgaggt tcgtcctgtg ggaagggaag ctgaggccgg tgccgtccaa gcccgccgac
cttccgttct tcgatctcat gagcatcccg ggcaagctcc gggccggcct tggcgccctc
ggcattcgcc cgccgcctcc agttcgttct ctccccaaat gctgcattcg tgattcttct
gcattttgat tgttcaactg tcattggang ttcgttctct ccccaaatgc tgcattcgtg
attcttctgc attttgattg ttcaactgtc attggcgctg agcgttactg gcaaaggttt
cagggccgtg aggagtcggt ggaggagttt gtgcgccgca acctcggtgc tgaggtcttt
gagcgcctca tcgagccttt ctgctcaggt gcttattgca ttgtacaatg gctgttttgt
tatgatttcc tgcttgcagt cattccctat aaaagaatgt aacagtttca atgttaacat
aaggtgtcta tgctggtgat ccttcaaaac tcagtatgaa ggctgcattt gggaaggtat
ggaggcttga ggaggctgga ggtagtatta ttggtggaac aattaaaaca atccaggaga
ggggcaagaa tcctaagcca gagagggatc cgtaagagaa caacttactt ttcttctgtt
gcattatctg tctattctta tcattctgat caattttctc cagccgcctt ccaaagccaa
agggacagac tgttgcatcc ttcaggaagg gtctcgccat gcttccaaat gccattacat
ccaggtttat tctcatatca tgtatatact atttgtatag cgcatcaact ttgcacgagg
gtgccacaga ttgtttggat agacgtagtc cttcagagac ttcgtacatc tcgtttgcca
aattcattac catattattg catcacctct tttcatcctc agtgcatatt agttaggtta
ctttcaccct ttttcatctc actttatgtt gtcatttaca atataggtaa atggtttcta
gttgtctgtg gaaactacta acatatatgg cttcagattg taagggataa atacagatta
tgcatttatt ctagtgtgtg tgcttgtgac ttcatcaaaa gctatgaaaa tttgcaattg
agatatctga tatagactta tggtactaac cacttaactt tgttccattc tgatccacct
ttgtttacag gttgggtagt aaagtcaaac tgtcttggaa actcacaagc attacaaaat
cagacagcaa gggttatgta ttggtgtatg aaacaccaga aggaatcgtc tcagtccagg
ctaaaagtgt tatcatgacc attccctcct acgttgctag tgacatcttg cgcccacttt
cagtaagtat aaaactaatc aaatttcatg ttctgtaaac tgaggcactg ttagcttctt
aattgggaaa tgggcaccca gcagtagcat tcacacttaa ggaattcatt ctttctgcca
ttctgtagtc aacgtctcaa tgcagagatg tatagaaaca caaaggaaat tgctacacct
tgtttgattt gagtaacatc tgaatgttta attttcagaa tggatcatct ttctgagata
tgcataatgt aacttaagcc tttcctaatt actaaacaca attgaattgc tgagattgaa
gagaagcttg tgcaccatca tttgagatgg cttgcacata tccaacagag gcctcaagat
gcactggtaa tgtgaacaag aggcagagga agaccaaact tgacattgga ggagtctgtg
aagagggata tgaaggactg gaatatcgtt aaggagttag ctatagatag agaacgtgta
aaccaataac taacgtgcca aaaccttgat tttcggtttc ccccattttt acccttttct
gtttggttta ttattgttct tttcctctcc ttgtttcttt ctttttttcc tttttgtgta
atctctttgt gtgagtcttg cgggtttcat ctctagtcta tcccaacttg ggactaaaag
gttttattgt tgttgttgaa ttcaattgct gagatcagaa ggattgaacg gaagaaggtt
atgcgaatgg aaatgcttta ggtcgagggc aaaagagtca taatattgat gtactagttc
ggtttggaca gcttcttaac caattaatgt tctaggcttt caaataatct gaaacaacaa
cgttttatgt ttctactgtt ttcctaagtt tatatcttgt tcgaacaaaa atggacgtag
tgatcaattt tgataattct atatgttcag gtaactgcta tataacttgt caactacaga
attggggtta ttcaagtatg agagaggaga ctatgtttag tagcactata caatttccta
gaatttctaa ggtaatgtag gcatacttaa cttgtttttc cataccacag agtgatgctg
cagatgctct ttcaagattc tattatccac cagtcgctgc tgtaactgtt tcatatccaa
aggaagcaat tcgaaaagaa tgcttaatcg atggggagct ccagggtttc ggccagttgc
atccacgtag tcaaggagtt gagacattag gtaaattttc cttttttttt acatttgagt
cttacgctct gtacagatca ttatcattat tatgtggctt aatgttttta ttgtatttgt
taaagtacta tttggattgc aaatcgacaa tcttattggt gttattcttt tcaaaggaac
aatatacagc tcttcgctct ttccgaatcg tgctcctgct ggaagggtct tacttcttaa
ctacatagga ggtgctacaa atacagggat cgtttccaag gttctattct ctgtcaaact
gattgatcct tatttgttca tgatcttctg atatgtttac aacatccttt actgtgatct
tcaaataaaa cacatgttac tgctgatcac ctattcttca ttgattgacg taaatgctga
ctgcattttt cactactgat ctcatcttat ttgcagagcg aaagtgagct ggtagaagca
gttgaccgtg atcttaggaa aatgctcata aatcctagag cagtggatcc ttatgtcctt
ggtgtgcggg tgtggccaca agctattcct cagttcctgg taggacatct tgatattctc
gatgctgcaa aatctgccct caacagaagt ggctacgatg ggctattcct gggagggaac
tatgtagcag gagttgctct ggggcgctgc gttgaaggtg catatgaaag tgcctcacaa
atatctgact tcttgaccaa gtacgcctac aagtga
PPO2 from Eleusine indica-mitochondrial (QHA79697.1; GI: 1788579950)
(SEQ ID NO: 3)
magsddtraa parsvavvga gvsglaaayr lrksgvnvtv feagdraggk irsnseggfl
wdegantmte selevsrlid dlglqdrqqy pnsqhkryiv kdgapvlips dpislikssv
lstkskfalf lepfiykkts trnsgivsde hlsesvgsff erhfgqevvd ylidpfvagt
sggdpeslsi rhafpalwnl ekkygsviag ailskltakr dpvkktsdss gkrrnrrvsf
sflggmqsli dalhnevgdg nvklstevls lacsvdgvpa sggwsisids kntgskefgk
kqafdavimt aplsnvqkmk fmkggapfvl dflpkvdylp lslmvtaykk edvkrplegf
gvlipykeqq khglktlgtl fssmmfpdra psdqflyttf iggshnrdla gapttilkql
vtsdlrkllg vegqptfvkh vywrnafply grdynsvlea iekmeknlpg ffyagnnkdg
lavgsviasg skaadlaisy lesrtkhdss h
PPO2 from Eleusine indica-mitochondrial (MK573538.1)
(SEQ ID NO: 4)
atggcgggct ccgacgacac gcgcgccgct cccgccaggt cggtcgccgt cgtgggcgcc
ggcgtcagtg ggctcgcggc ggcgtacagg ctgcggaaga gcggcgtcaa cgtgacggtg
ttcgaggcgg gtgacagggc cggagggaag atacgaagca actccgaggg tggattcctc
tgggatgaag gggccaacac catgacagaa agtgaattgg aggtcagcag attaattgat
gatcttggtc tccaagacag acagcagtat cctaactccc aacacaagcg ttacattgtc
aaagatggtg caccagtact gattccttca gatcctattt ccttaataaa aagcagtgtt
ctgtctacaa aatcaaagtt tgcattattt ctagagccat ttatttacaa gaagactagc
acaaqaaact ctqqaataqt qtctqatqaq catttaaqtq agaqtqttgq qaqcttcttt
gaacgccact ttggacaaga ggtagttgac tatcttatcg atccatttgt agctggaaca
agtggcggag atcctgagtc attatctatt cgacatgcat ttccagcact gtggaattta
gagaaaaagt atggttctgt cattgctggc gccatcttgt ctaaactaac tgccaaacgc
gatcctgtca agaaaacaag tgattcatca gggaaaagaa ggaataggcg tgtgtcattt
tcatttcttg gaggaatgca gtcactaata gatgcacttc acaatgaagt tggagatggt
aatgtgaagc tcagtacaga agtgttgtcc ctggcatgta gtgtcgatgg tgtgcctgca
tccggtgggt ggtcaatttc tattgattca aaaaataccg gtagcaagga gtttggaaag
aaacaagcct tcgatgctgt aataatgaca gctccattgt ctaatgtgca gaagatgaag
tttatgaaag ggggagctcc atttgtgtta gactttcttc ctaaggtgga ttatctgcca
ctatccctca tggtgacagc ttacaagaag gaagacgtca agagacctct ggaaggattt
ggggtactaa taccctataa ggaacagcaa aagcatggcc tgaaaactct tggtactctc
ttctcctcga tgatgttccc agatcgagct cctagtgatc aatttctata tacaactttc
attgggggta gccacaatcg agatcttgct ggagctccaa cgactattct aaagcaactt
gtgacatctg accttagaaa gcttttgggt gtggaagggc aaccaacttt tgtcaagcat
gtatactgga gaaatgcttt tcctttgtat ggccgtgatt acaattccgt attggaagct
atagagaaga tggagaaaaa tctaccaggg ttcttctatg caggaaataa caaggatggg
ctggctgttg ggagtgttat agcttctgga agcaaggctg ctgaccttgc aatttcgtat
cttgaatctc gcacaaagca tgacagttca cattgaa
PPO from Eleusine indica-codon optimized
(SEQ ID NO: 5)
ATGGTTGCAACACCTGCTATGGCTGCAGCTGCACCACCTTTGAGGGCACCAAGATTTCATGCTAGGCACAGAAG
AAGGTCTGTTAGATGTGCTGTTGCATCAGATGCAACCGAGGCTCCAGCTGCACCTGGTGCAAGGTTGTCTGCTG
ATTGTGTTGTGGTTGGAGGTGGAATTTCAGGATTGTGCACAGCTCAAGCACTTGCTACCAAGCATGGTATTGGA
GATGTGCTTGTTACTGAAGCAAGGGCTAGACCAGGTGGAAACATTACTACAGTGGAGAGACCTGATGAAGGTTA
TTTGTGGGAGGAAGGACCAAATTCTTTCCAGCCATCAGATCCTGTGCTTACAATGGCTGTTGATTCTGGTTTGA
AGGAAGATTTGGTTTTTGGAGATCCAAACGCACCTAGGTTCGTGTTGTGGGAGGGAAAGTTGAGACCAGTTCCT
TCTAAGCCAGCTGATTTGCCATTTTTCGATCTTATGTCAATTCCTGGAAAGTTGAGGGCAGGTTTGGGAGCTTT
GGGTATTAGGCCACCTCCACCTGGAAGAGAGGAATCTGTGGAGGAATTTGTTAGGAGAAATTTGGGTGCTGAGG
TGTTTGAAAGACTTATTGAGCCTTTCTGCTCAGGTGTTTACACCGGAGATCCATCTAAGTTGTCTATGAAGGCT
GCATTCGGAAAGGTTTGGAGGCTTGAGGAAGCTGGTGGATCTATTATTGGTGGAACAATTAAGACCATTCAAGA
GAGAGGAAAGAACCCAAAGCCTGAAAGAGATCCAAGACTTCCAAAGCCTAAGGGTCAGACCGTGGCTTCTTTTA
GAAAGGGATTGGCAATGCTTCCAAATGCTATTACTTCTAGGCTTGGTTCAAAGGTTAAGTTGTCTTGGAAGTTG
ACTTCAATTACAAAGTCAGATTCTAAGGGTTATGTGCTTGTTTACGAGACTCCAGAAGGAATTGTGTCTGTTCA
AGCAAAGTCAGTGATTATGACAATCCCTTCTTATGTTGCTTCAGATATTTTGAGGCCACTTTCTTCAGATGCTG
CAGATGCTCTTTCTAGGTTCTATTACCCACCTGTGGCTGCAGTGACTGTTTCATACCCTAAGGAAGCTATTAGG
AAGGAATGTTTGATTGATGGAGAGCTTCAAGGTTTTGGACAGCTTCATCCAAGGTCTCAGGGTGTTGAAACCTT
GGGAACTATCTATTCTTCTTCTCTTTTCCCAAACAGGGCACCTGCTGGTAGAGTGCTTTTGCTTAACTACATTG
GTGGAGCTACCAATACTGGAATTGTTTCAAAGTCTGAGTCAGAATTGGTGGAAGCAGTTGATAGGGATTTGAGA
AAGATGCTTATTAATCCAAGGGCTGTTGATCCTTATGTTTTGGGTGTGAGAGTTTGGCCACAAGCAATTCCTCA
GTTTCTTGTGGGACACTTGGATATTCTTGATGCTGCAAAGTCTGCTCTTAACAGGTCAGGATACGATGGATTGT
TCCTTGGTGGAAATTACGTGGCAGGTGTTGCTTTGGGAAGATGCGTTGAGGGAGCATACGAATCTGCTTCACAA
ATTTCTGATTTTCTTACAAAGTATGCTTACAAG

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A plant comprising a mutation in a gene encoding a polypeptide having protoporphyrinogen IX oxidase activity, wherein the mutation comprises a substitution of an alanine (A) to a threonine (T) at residue 212 (relative to SEQ ID NO:1 when aligned using BLAST) and imparts a phenotype of herbicide resistance to the plant, wherein the herbicide is an oxadiazole.

2. The plant of claim 1, wherein the plant is selected from wheat, corn, soybean, tobacco, brachiaria, rice, millet, barley, tomato, apple, pear, strawberry, orange, alfalfa, cotton, carrot, potato, sugar beets, yam, lettuce, spinach, petunia, rose, chrysanthemum, turf grass, pine, fir, spruce, heavy metal accumulating plants, sunflower, safflower, rapeseed, and Arabidopsis.

3. The plant of claim 1, wherein the mutation is a point mutation.

4. The plant of claim 1, wherein the herbicide is oxadiazon.

5. Seed produced from the plant of claim 1.

6. Progeny of the plant of claim 1 any of claims 1-4.

7. A method of making a herbicide-resistant plant, comprising the steps of:
a) mutagenizing plant cells;
b) obtaining one or more plants from the cells; and
c) identifying at least one of the plants that contains a mutation in a gene encoding a polypeptide having a wild-type sequence as shown in SEQ ID NO:1 and exhibiting protoporphyrinogen IX oxidase activity, wherein the mutation comprises a substitution of an alanine (A) to a threonine (T) at residue 212 (relative to SEQ ID NO:1 when aligned using BLAST) and imparts a phenotype of herbicide resistance to the plant, wherein the herbicide is an oxadiazole.

8. The method of claim 7, wherein the mutagenizing utilizes a chemical mutagen, ionizing radiation, or fast neutron bombardment.

9. The method of claim 7, wherein the mutagenizing step comprises CRISPR, TALEN, or zinc-finger nuclease.

10. The method of claim 7, wherein the plant cells are selected from wheat, corn, soybean, tobacco, brachiaria, rice, millet, barley, tomato, apple, pear, strawberry, orange, alfalfa, cotton, carrot, potato, sugar beets, yam, lettuce, spinach, petunia, rose, chrysanthemum, turf grass, sunflower, safflower, rapeseed, and Arabidopsis.

11. The method of claim 7, wherein the plant cells are in a seed.

12. The method of claim 7, wherein the mutagenizing step is performed on seed from the plant.

13. The method of claim 7, wherein the mutation is a point mutation.

14. The method of claim 7, wherein the herbicide is oxadiazon.

15-20. (canceled)

21. A nucleic acid operably linked to a heterologous promoter, wherein the nucleic acid encodes a protoporphyrinogen IX oxidase (PPO) having a threonine at position 212 (relative to SEQ ID NO:1 when aligned using BLAST).

22. The nucleic acid of claim 21, wherein the nucleic acid has at least about 50% sequence identity to SEQ ID NO:2.

23. A vector comprising the nucleic acid of claim 21.

24. The vector of claim 23, wherein the vector is a plant transformation vector.

25. A host cell comprising the nucleic acid of claim 21.

26. The host cell of claim 25, wherein the host cell is a bacterial cell or a plant cell.

27-36. (canceled)
